(19) United States
(12) Patent Application Publication
(10) Pub. No.: US 2022/0265830 A1
(43) Pub. Date: Aug. 25, 2022

(54) LONG-ACTING FORMULATIONS AND VEHICLES

(71) Applicant: DURECT CORPORATION, Cupertino, CA (US)

(72) Inventors: Susan AUTIO, San Jose, CA (US); Keith Edward BRANHAM, Pelham, AL (US); James A. FILICE, Los Gatos, CA (US); John W. GIBSON, Springville, AL (US); John J. LEONARD, Morgan Hill, CA (US); James MATRIANO, Mountain View, CA (US); Whitney MORO, Birmingham, AL (US); Michael SEKAR, Palo Alto, CA (US); Chelsea Alexandra SNYDER, San Mateo, CA (US); Robert G. STRICKLEY, San Mateo, CA (US); Raju SUBRAMANIAN, San Mateo, CA (US); Felix THEEUWES, Los Altos Hills, CA (US); Monica TIJERINA, Foster City, CA (US); Jeremy C. WRIGHT, Los Altos, CA (US); Su II YUM, Los Altos, CA (US); Faye XU, San Jose, CA (US)

(73) Assignee: DURECT CORPORATION, Cupertino, CA (US)

(21) Appl. No.: 17/627,512

(22) PCT Filed: Jul. 17, 2020
(86) PCT No.: PCT/US2020/042605

§ 371 Date: Jan. 14, 2022

Related U.S. Application Data
(60) Provisional application No. 62/875,795, filed on Jul. 18, 2019.

(51) Int. Cl. A61K 47/26; A61K 45/06; A61K 47/22; A61K 47/34; A61K 9/08

(52) U.S. Cl. CPC A61K 47/26; A61K 45/06; A61K 47/22; A61K 47/34; A61K 9/08

(57) ABSTRACT

The present disclosure provides long-acting formulations and vehicles, methods of making the same and methods of using the same.

### CROSS REFERENCE TO RELATED APPLICATIONS

This application claims priority to and benefit of U.S. Application No. 62/875,795, filed Jul. 18, 2019, the entire contents of which are hereby incorporated by reference in their entirety.

### JOINT RESEARCH AGREEMENT

The claimed invention was made by or on behalf of one or more of the following parties to a joint research agreement: Durect Corporation and Gilead Sciences, Inc. The agreement was in effect on or before the effective filing date of the claimed invention, and the claimed invention was made as a result of activities undertaken within the scope of the agreement.

### BACKGROUND

Less complicated and less frequent dosing regimens can be advantageous for patients, healthcare providers, and for public health in general. Administration of long-acting medications has several benefits over short-acting oral tablets, e.g., improved convenience and increased compliance, resulting in, e.g., fewer relapses, hospital visits, and healthcare costs.

### SUMMARY

Less complicated dosing and less frequent dosing is advantageous. Long-acting medications have several advantages over short-acting oral tablets when administered, e.g., assurance of compliance resulting in fewer relapses and re-hospitalizations.

Some long-acting therapies require a loading dose when the therapy is initiated to achieve a good release profile. A loading dose is an extra dose that is given early in a treatment regimen to compensate for inadequate control over plasma level before a sustained release formulation achieves steady state. Loading doses may be delivered orally or by injection. Loading doses are undesirable as they may lead to additional anxiety, agitation, or lack of compliance with therapy. There remains, however, a need for compositions and methods that provide reproducible, controlled delivery of pharmaceutical active agents with low toxicity. Accordingly, there also remains a need for methods of making these compositions that provide reproducible, controlled delivery of pharmaceutical active agents with low toxicity.

The present disclosure provides long-acting formulations of active agents, and furthermore demonstrates that provided formulations can achieve particular desirable results (e.g., extended release). Long-acting formulations permit less frequent dosing schedules, which, e.g., can increase patient compliance with antiviral therapy (often comprising multiple drug products administered according to various dosing regimens). For instance, patient compliance with antiviral therapy leads to increased efficacy and limits the possibility of developing a resistant viral strain.

In some embodiments, provided compositions comprise an active agent and a vehicle comprising a high viscosity liquid carrier material (HVLCM), e.g., sucrose acetate isobutyrate (SAIB). Provided compositions may further comprise a polymer (e.g., poly(lactic acid)(glycolic acid)) and/or a solvent (e.g., propylene carbonate). In some embodiments, provided compositions comprise surprisingly small amounts of water.

In some embodiments, provided compositions comprise an active agent, or a pharmaceutically acceptable salt thereof, and sucrose acetate isobutyrate. In some embodiments, provided compositions comprise (i) an active agent or a pharmaceutically acceptable salt thereof; (ii) sucrose acetate isobutyrate; and (iii) propylene carbonate. In some embodiments, provided compositions comprise (i) an active agent or a pharmaceutically acceptable salt thereof; (ii) sucrose acetate isobutyrate; (iii) propylene carbonate; and (iv) poly(lactic acid)(glycolic acid).

In some embodiments, provided compositions have one or more desirable characteristics, including but not limited to resistance to phase separation, suitable viscosity, stability upon storage, and/or suitable release profile. In some embodiments, provided compositions display a suitable release profile, e.g., a release profile that is sustained at a particular level over a particular period of time and/or that does not display an initial burst release of an active agent.

The present disclosure also provides methods of manufacturing provided compositions, comprising providing a vehicle comprising a HVLCM; and combining the vehicle with an active agent under suitable conditions to give the provided composition.

The present disclosure also provides methods of administering compositions and dosage forms provided herein.

### BRIEF DESCRIPTION OF THE DRAWINGS

FIG. 1 shows the cumulative release (%) of tenofovir alafenamide (TAF) from selected formulations of Table 13. For formulation F5 in FIG. 1, Dulbecco's PBS pH 7.4 buffer was used for the first 10 days. For all other time points in FIG. 1, 20 mM KH₂PO₄, pH 6.0, with 0.9% NaCl buffer was used.

FIG. 2 depicts the delivery rate (μg/h) of TAF from selected formulations of Table 13. For formulations F4, F5, F6, F7 and F10 in FIG. 2, Dulbecco's PBS pH 7.4 buffer was used for the first 10 days. For all other time points in FIG. 2, 20 mM KH₂PO₄, pH 6.0, with 0.9% NaCl buffer was used.

FIG. 3 shows the cumulative release (%) of TAF from selected formulations of Table 13. For formulations F4 and F5 in FIG. 3, Dulbecco's PBS pH 7.4 buffer was used for the first 10 days. For all other time points in FIG. 3, 20 mM KH₂PO₄, pH 6.0, with 0.9% NaCl buffer was used.

FIG. 4 depicts the delivery rate (μg/h) of TAF from selected formulations of Table 13 in 20 mM KH₂PO₄, pH 6.0, with 0.9% NaCl buffer.

FIG. 5 shows the cumulative release (%) of TAF from additional selected formulations of Table 13. For formulations F4 and F5 in FIG. 5, Dulbecco's PBS pH 7.4 buffer was used for the first 10 days. For all other time points in FIG. 5, 20 mM KH₂PO₄, pH 6.0, with 0.9% NaCl buffer was used.

FIG. 6 shows the cumulative release (%) of a small molecule for treatment of Alzheimer's disease in aqueous buffer (pH˜4.7+0.5% SDS (ammonium acetate) at 37° C.) from selected formulations.

FIG. 7 shows the PK profile of a small molecule for treatment of Alzheimer's disease in dogs from formulation A1, a composition comprising sucrose acetate isobutyrate, N-methyl-pyrrolidone, and a poly(lactic acid)(glycolic acid) (PLGA).

FIG. 8 shows the PK profile of a small molecule for treatment of Alzheimer's disease in dogs from formulation A7, a composition comprising sucrose acetate isobutyrate, propylene carbonate, and a 75:25 poly(lactic acid)(glycolic acid) (PLGA).

FIG. 9 shows the PK profile of a small molecule for treatment of Alzheimer's disease in dogs from formulation A16, a composition comprising sucrose acetate isobutyrate, propylene carbonate, and a 90:10 poly(lactic acid)(glycolic acid) (PLGA).

FIG. 10 shows cumulative release (%) of TAF from selected formulations of Table 3B over a 2-day period.

FIG. 11 shows cumulative release (%) of TAF from selected formulations of Table 3B over a 24-day period.

FIG. 12 shows cumulative release (%) of risperidone from selected formulations of Table 19 over a 44-day period.

FIG. 13 shows cumulative release (%) of naltrexone from selected formulations of Table 24 over a 39-day period.

### DETAILED DESCRIPTION

### Definitions

The term “about” or “approximately”, when used herein in reference to a value, refers to a value that is similar, in context to the referenced value. In general, those skilled in the art, familiar with the context, will appreciate the relevant degree of variance encompassed by “about” in that context.

As used herein, the term “administering” or “administration” typically refers to the administration of a composition to a subject to achieve delivery of an agent that is, or is included, in a composition to a target site or a site to be treated. Those of ordinary skill in the art will be aware of a variety of routes that may, in appropriate circumstances, be utilized for administration to a subject, for example a human. For example, in some embodiments, administration may be parenteral. In some embodiments, administration may be by injection (e.g., intramuscular, intravenous, or subcutaneous injection). In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time). In some embodiments, administration may involve continuous dosing (e.g., perfusion) for at least a selected period of time.

As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc., to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the term “dosage form” refers to a physically discrete unit of an active agent (e.g., a therapeutic, prophylactic, or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a prophylactic or therapeutic dosing regimen). Those of ordinary skill in the art appreciate that the total amount of a composition or agent administered to a particular subject is determined by one or more attending physicians and may involve administration of multiple dosage forms.

The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and/or animals without undue toxicity, irritation, allergic response and the like, and are commensurate with a reasonable benefit/risk ratio. Pharmaceutically acceptable salts are well known in the art. For example, S. M. Berge, et al. describes several pharmaceutically acceptable salts in detail in J. Pharmaceutical Sciences, 66: 1-19 (1977), which is hereby incorporated by reference in its entirety.

As used herein, the term “subject” refers to an organism, typically a mammal (e.g., a human). In some embodiments, a subject is suffering from a relevant disease, disorder or condition. In some embodiments, a human subject is an adult, adolescent, or pediatric subject. In some embodiments, a subject is susceptible to a disease, disorder, or condition. In some embodiments, a subject is healthy. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy and/or prophylaxis is and/or has been administered.

As used herein, “therapeutically effective amount” is an amount that produces the desired effect for which it is administered. In some embodiments, the term “therapeutically effective amount” or “therapeutically effective dose” means an amount that is sufficient, when administered to a population suffering from or susceptible to a disease, disorder, and/or condition in accordance with a therapeutic dosing regimen, to treat or prevent the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment or prevention be achieved in a particular individual. Rather, a therapeutically effective amount may be that amount that provides a particular desired pharmacological response in a significant number of subjects when administered to patients in need of such treatment or prevention. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### Provided Compositions:

Provided herein are novel compositions that comprise and/or deliver an active agent and are formulated for controlled release (i.e., a long-acting formulation). Provided compositions are useful in methods described herein.

### Components of Provided Compositions

### Active Pharmaceutical Agent:

Provided compositions comprise an active agent.

In one aspect, the pharmaceutical active agent comprises at least one member selected from peptide, protein, antibody, carbohydrate, small molecule, nucleic acid, and nucleoside.

Representative pharmaceutical active agents include drug, peptide, protein, carbohydrate (including monosaccharides, oligosaccharides, and polysaccharides), nucleoprotein, mucoprotein, lipoprotein, synthetic polypeptide or protein, or a small molecule linked to a protein, antibody, glycoprotein, steroid, nucleic acid (any form of DNA, including cDNA, or RNA, or a fragment thereof), nucleotide, nucleoside, oligonucleotides (including antisense oligonucleotides), gene, lipid, hormone, mineral supplement, vitamin including vitamin C and vitamin E, or combinations of any of the above, that cause(s) a biological effect when administered in vivo to an animal, including but not limited to birds and mammals, including humans.

Drug means any substance used internally or externally as a medicine for the treatment, cure, or prevention of a disease or disorder, and includes but is not limited to immunosuppressants, antioxidants, anesthetics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers.

In one embodiment disclosed herein, the pharmaceutical active agent is a vaccine and the substance to be delivered is an antigen. The antigen can be derived from a cell, bacteria, or virus particle, or portion thereof. As defined herein, antigen may be a protein, peptide, polysaccharide, glycoprotein, glycolipid, nucleic acid, or combination thereof, which elicits an immunogenic response in an animal, for example, a mammal, bird, or fish. As defined herein, the immunogenic response can be humoral or cell-mediated. In the event the material to which the immunogenic response is to be directed is poorly antigenic, it may be conjugated to a carrier such as albumin or to a hapten, using standard covalent binding techniques, for example, with one of the several commercially available reagent kits.

Examples of antigens include viral proteins such as influenza proteins, human immunodeficiency virus (HIV) proteins, and hepatitis A, B, or C proteins, and bacterial proteins, lipopolysaccharides such as gram negative bacterial cell walls and Neisseria gonorrhea proteins, and parvovirus.

Other examples include HPV (human papiloma virus) vaccine, peptide vaccines, whole vaccines, subunit vaccines, oligosaccharides, lipoproteins. Examples of subunit vaccines include: Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glyconconjugate, Group A polysaccharide with tetanus toxoid M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

The active agent may include one or more of whole virus and bacteria, which include weakened or killed viruses such as: Cytomegalo virus, Hepatitis B virus, Hepatitis C virus, Human papillomavirus, Rubella virus, and Varicella zoster.

The active agent may include one or more of weakened or killed bacteria such as: Bordetella pertussis, Clostridium tetani, Corynebacterium diptheriae, Group A streptococcus, A streptococcus, Legionella pneumophila, Neisseria meningitdis, Pseudomonas aeruginosa, Streptococcus pneumonia, Treponema pallidum, and Vibrio cholerae and mixtures thereof.

The active agent may include one or more vaccine that contain an antigenic agent such as: Flu vaccines, Lyme disease vaccine, Rabies vaccine, Measles vaccine, Mumps vaccine, Chicken pox vaccine, Small pox vaccine, Hepatitis vaccine, Pertussis vaccine, Diptheria vaccine, Recombinant protein vaccines, DNA vaccines, and Therapeutic cancer vaccines.

Non-limiting examples of pharmaceutical active agents include anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; vaccines such as smallpox, yellow fever, distemper, hog cholera, chicken pox, antivenom, scarlet fever, dyptheria toxoid, tetanus toxoid, pigeon pox, whooping cough, influenzae, rabies, mumps, measles, poliomyelitic, and Newcastle disease; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl) urea, carbromal; psychic energizers such as 3-(2-aminopropyl) indole acetate and 3-(2-aminobutyl) indole acetate; tranquilizers such as reserpine, chlorpromayline, and thiopropazate; androgenic steroids such as methyl-testosterone and fluorymesterone; estrogens such as estrone, 17-.beta.-estradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-.beta.-hydroxy-progesterone; humoral agents such as the prostaglandins, for example PGE₁, PGE₂ and PGF₂; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; antipsychotics including typical and atypical antipsychotics, wherein the atypical antipsychotics comprise risperidone, paliperidone, or olanzapine; nutritional agents such as vitamins, natural and synthetic bioactive peptides and proteins, including growth factors, cell adhesion factors, cytokines, and biological response modifiers; together with pharmaceutically acceptable salts of the above.

The pharmaceutical active agent is typically included in the composition in an amount sufficient to deliver to the host animal or plant an effective amount to achieve a desired effect. The amount of pharmaceutical active agent incorporated into the composition depends upon the desired release profile, the concentration of pharmaceutical active agent required for a biological effect, and the desired period of release of the pharmaceutical active agent.

The concentration of pharmaceutical active agent in the composition will also depend on absorption, inactivation, and excretion rates of the pharmaceutical active agent as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the disclosed compositions, and that the concentration ranges set forth herein are exemplary only and are not intended to limit the scope or practice of the present disclosure. The compositions may be administered in one dosage, or may be divided into a number of smaller doses to be administered at varying intervals of time.

In some cases, the pharmaceutical active agent comprises an antipsychotic, such as an atypical antipsychotic. Examples of anti-psychotic drugs include, but are not limited to metabotropic glutamate receptor 2 agonists, glycine transporter 1 inhibitors, partial agonists of dopamine receptors, chlorpromazine, fluphenazine, mesoridazine, perphenazine, prochlorperazine, promazine, thioridazine/sulforidazine, trifluoperazine, butyrophenones (azaperone, benperidol, droperidol, haloperidol), thioxanthenes (flupentixol, chlorprothixene, thiothixene, zuclopenthixol), diphenylbutylpiperidines (fluspirilene, penfluridol, pimozide, loxapine), butyrophenones (melperone), indoles (sertindole, ziprasidone, molidone), benzamides (sulpiride, remoxipride, amisulpride), diazepines/oxazepines/thiazepines (clozapine, olanzapine, quetiapine), aripiprazole, risperidone, paliperidone, zotepine), amisulpride, asenapine, iloperidone, lurasidone, cannabidiol, tetraenazine, and L-theanine, including pharmaceutically acceptable salts, solvates, bases, and ester forms thereof. Combinations of two or more of these compounds, or combinations with other compounds are included in the scope of the disclosure.

For instance, the active agent may comprise at least one member selected from chlorpromazine, fluphenazine, mesoridazine, perphenazine, prochlorperazine, promazine, thioridazine, sulforidazine, trifluoperazine, molindone, azaperone, benperidol, droperidol, haloperidol, flupentixol, chlorprothixene, thiothixene, zuclopenthixol, fluspirilene, penfluridol, pimozide, loxapine, melperone, sertindole, ziprasidone, sulpiride, remoxipride, amisulpride, clozapine, olanzapine, quetiapine, aripiprazole, risperidone, paliperidone, zotepine, amisulpride, asenapine, iloperidone, lurasidone, cannabidiol, tetraenazine, and L-theanine, or pharmaceutically acceptable salt thereof.

In some embodiments, the active agent comprises an HIV drug such as stavudine, rilpivirine, saquinavir, dolutegravir, elviteqravir, raltegravir, zalcitibine, pharmaceutically acceptable salts thereof, and mixtures thereof.

In some embodiments, the active agent comprises an Alzheimer's drug such as nemantine, donepezil, brexpiprazole, pharmaceutically acceptable salts thereof, and mixtures thereof.

In some embodiments, the active agent comprises a Parkinson's drug such as levodopa, carbidopa, glatiramer, pharmaceutically acceptable salts thereof, and mixtures thereof.

In some embodiments, the active agent comprises a drug for drug dependance or addiction control such as naltrexone, buprenorphine, naloxone, pharmaceutically acceptable salts thereof, and mixtures thereof.

In some embodiments, the active agent comprises a cancer drug (e.g., a leukemia drug) such as decitabine, azacitidine, leuprolide, pharmaceutically acceptable salts thereof, and mixtures thereof.

In some embodiments, the active agent comprises a diabetes drug such as exenatide or liraglutide pharmaceutically acceptable salts thereof, and mixtures thereof.

In some embodiments, the active agent comprises a pharmaceutically acceptable salt. Exemplary salts include hydrochloride, phosphate, citrate, maleate, mesylate, pamoate, and naphthaline-2-sulfonate monohydrate. For instance, representative salts include pamoate and naphthaline-2-sulfonate. In some cases, the salt is lipophilic. An exemplary ester is palmitate.

Factors generally considered when selecting the active agent include (1) need for long acting injectable products; (2) subcutaneous and/or IM dosing possibility; (3) a subcutaneous dosing mass typically of 3 to 5 g at most or an IM dosing mass typically of 5 to 10 g at most; (4) drug concentration in the composition typically of 30-50 wt % at most; (5) injection site tolerance acceptable; and (6) controlled release capability by solution-diffusion mechanisms and drug stabilizing attributes.

The active agents shown below are candidates, in part, because of their biopharmaceutical classification system (BCS) class, oral half-life, water solubility, log P, and/or pKa.

| | | | | Experimental [Predicted] | | |
|---|---|---|---|---|---|---|
| | | BCS | Oral | Water | | |
| Indication | Generic Name | Class | Half-life | Solubility | logP | pKa |
| HIV drugs | Stavudine | 1, 3 | 0.8-1.5 hrs | 5-10 g/100 mL at | −0.72 | [9.95] |
| | | | | 21° C. | | |
| | Rilpivirine | | 34-55 hrs | <0.1 mg/mL | 4.86 | 5.6 |
| | Saquinavir | | | [0.00247 | 3.8 | [13.61, |
| | | | | mg/mL] | | 8.47] |
| | Dolutegravir | | 14 hrs | Slightly soluble | 2.2 | 8.2 |
| | | | | [0.0922 mg/mL] | | |
| | Raltegravir | | 9 hrs | | [−0.39] | [5.62] |
| | Elvitegravir | | 8.7 hrs | <0.3 meg/mL | [3.66, | [6.16] |
| | | | | [0.00652 | 4.67] | |
| | | | | mg/mL] | | |
| | Zalcitabine | | 2 hrs | 7.64E+004 mg/L | −1.30 | [14.67] |
| | | | | (at 25° C.) | | |
| | | | | [7.05 mg/mL] | | |
| Alzheimer | Nemantine | | | | | |
| | Donepezil | 1 | 70 hrs | 2.931 mg/mL | 3.6 | |
| | | | | [0.0045 mg/mL] | | |
| | Brexpiprazoie | | 91 hrs | [0.00227 | [5.38, | [13.56, |
| | | | | mg/mL] | 4.65] | 8.4] |
| Parkinson's | Carbidopa/ | 1 | ~107 min | 3.8 mg/mL | −1.9 | 2.3 |
| and/or Multiple | Levodopa | 3 | 50 min | 5 mM | 0.05 [−2.3, | [1.65, |
| Sclerosis | Glatiramer | | | [3.3 mg/mL] | −1.8] | 9.06] |
| Drug | Naltrexone | | 4 hrs | 100 mg/mL as | 1.92 | [7.39, |
| dependence or | | | | HC salt [3.07 | | 11.54] |
| addiction | | | | mg/mL] | | |
| control | Buprenorphine | 3 | IV 2.2 hrs | [0.0168 mg/mL] | 4.98 | 8.31 (at |
| | | | | | | 25° C.) |
| | Naloxone | 3 | IM 1.24 | [5.64 mg/mL] | 2.09 | [10.07, |
| | | | hrs | | | 7.84] |
| Cancers | Decitabine | | 0.51 ± 0.31 | [5.5 mg/mL] | [−2.2] | [13.89, |
| (leukemia, etc.) | | | hrs | | | −0.25] |
| | Azacitidine | | ~4 hrs | 8.9E+004 mg/L | −3.5 | [12.55, |
| | | | | [12.1 mg/mL] | | −0.38] |
| | Leuprolide | | ~3 hrs | | | |
| CNS | Asenapine | | ~24 hrs | | [3.72] | [7.29, 1] |
| (Schizophrenia, | Risperidone | 2 | 3 hours up | 2.33 mg/mL | [3.27, | [8.76] |
| etc.) | | | to 20 | [0.171 mg/mL] | 2.63] | |
| | | | hours | | | |
| | Olanzapine | 2 | 21 −54 hrs | [0.0942 mg/mL] | 4.094 | 10.57 |
| | Paliperidone | | ~23 hrs | [0.297 mg/mL] | 1.8 | [13.74, |
| | | | | | | 8.76] |
| | Aripiprazole | 2 | 75 hrs | 0.00001% | [5.21, 4.9] | 7.6 |
| | | | | [0.00777 | | |
| | | | | mg/mL] | | |
| | Lurasidone | | 18-37 hrs | [0. 00789 | [5.25, | [8.5, 1] |
| | | | | mg/mL] | 4.56] | |
| Diabetes | Exenatide | | 2.4 hrs | | | PI = 4.86 |
| | Liraglutide | | 13 hrs | | | PI = 4.9 |

As discussed in more detail elsewhere in the present disclosure, acid-labile active agents (e.g., active agents having at least one pKa<9, such as <8, <7, <6, or <5) could benefit from the present compositions. While not wishing to be bound by theory, examples of acid-labile active agents include stavudine, rilpivirine, saquinavir, zalcitibine, nemantine, donepezil, brexpiprazole, levodopa, carbidopa, glatiramer, naltrexone, buprenorphine, naloxone, decitabine, azacitidine, leuprolide, risperidone, olanzapine, paliperidone, aripiprazole, lurasidone, and pharmaceutically acceptable salts thereof.

In some embodiments, provided compositions comprise about 1 wt %, about 2 wt %, about 5 wt %, about 8 wt %, about 10 wt %, about 12 wt %, about 15 wt %, about 18 wt %, about 20 wt %, about 25 wt %, about 30 wt %, about 40 wt %, or about 50 wt % active agent, based on the weight of the vehicle or the total weight of the composition. In some embodiments, provided compositions comprise from about 1 wt % to about 50 wt %, about 2 wt % to about 40 wt %, about 5 wt % to about 30 wt %, about 10 wt % to about 25 wt %, about 10 wt % to about 20 wt %, about 5 wt % to about 10 wt %, about 5 wt % to about 15 wt %, about 8 wt % to about 18 wt %, about 5 wt % to about 20 wt %, about 10 wt % to about 15 wt %, or about 10 wt % to about 12 wt % active agent, based on the weight of the vehicle or the total weight of the composition.

In some embodiments, provided compositions comprise about 1 wt %, about 2 wt %, about 5 wt %, about 8 wt %, about 10 wt %, about 12 wt %, about 15 wt %, about 18 wt %, about 20 wt %, about 25 wt %, about 30 wt %, about 40 wt %, or about 50 wt % of active agent, based on the weight of the vehicle or the total weight of the composition. In some embodiments, provided compositions comprise from about 1 wt % to about 50 wt %, about 2 wt % to about 40 wt %, about 5 wt % to about 30 wt %, about 10 wt % to about 25 wt %, about 10 wt % to about 20 wt %, about 5 wt % to about 10 wt %, about 5 wt % to about 15 wt %, about 8 wt % to about 18 wt %, about 5 wt % to about 20 wt %, about 10 wt % to about 15 wt %, or about 10 wt % to about 12 wt % active agent, based on the weight of the vehicle or the total weight of the composition.

It will be understood that compositions or formulations comprising an active agent may comprise the active agent in one of several forms (e.g., free base form, salt form, etc.). It will be understood, therefore, that reference to an amount (e.g., in mg or wt %) of the active agent means the amount of active agent in free base form. Accordingly, an active agent may be provided and/or utilized as, e.g., a salt form of an active agent such that the amount of the salt (or other form) in an amount that corresponds to the “free base equivalent” of the active agent. For example, in the case of tenofovir, “25 mg tenofovir alafenamide” means, e.g., approx. 35.6 mg of tenofovir alafenamide sebacate, approx. 35.2 mg tenofovir alafenamide hemipamoate, etc.

Without wishing to be bound by any particular theory, salt forms of active agents that are poorly soluble may be particularly useful in provided compositions. Furthermore, the present disclosure encompasses the recognition that compositions comprising both a long-acting salt of an active agent (e.g., a poorly soluble salt) and a HVLCM, as described herein, are particularly effective as long-acting formulations. Accordingly, in some embodiments, provided compositions comprise an active agent as a pharmaceutically acceptable salt, wherein the active agent salt has a solubility of less than about 5 mg/mL, less than about 2 mg/mL, or less than about 1 mg/mL in deionized water at about 22° C. In some embodiments, provided compositions comprise an active agent as a pharmaceutically acceptable salt, wherein the active agent salt has a solubility of less than about 10 mg/mL, less than about 5 mg/mL, less than about 2 mg/mL, or less than about 1 mg/mL in the composition at about 25° C. In some embodiments, provided compositions comprise an active agent as a pharmaceutically acceptable salt, wherein the active agent salt has a solubility of about 0.2 mg/mL to about 10 mg/mL, about 0.5 mg/mL to about 8 mg/mL, about 1 mg/mL to about 6 mg/mL, or about 2 mg/mL to about 5 mg/mL in the composition at about 25° C.

In some embodiments, the active agent is dissolved or suspended in the composition. Particles comprising the active agent, which are used to make the provided compositions, typically have a median particle size, as measured by laser diffraction, from about 0.1 μm to about 100 μm, from about 0.2 μm to about 90 μm, from about 0.25 μm to about 80 μm, from about 0.5 μm to about 70 μm, from about 1 μm to about 70 μm, from about 2 μm to about 60 μm, from about 5 μm to about 60 μm, from about 10 μm to about 50 μm, or from about 10 μm to about 40 μm.

In the context of the present disclosure, the median particle size, as measured by laser diffraction, refers to the size of the particles before addition with the vehicle. Thus, the recited compositions are “made from” or “obtainable by combining” the particles comprising the pharmaceutical active agent and the one or more further specified components.

### Vehicle:

In some embodiments, the present disclosure provides compositions comprising an active agent and a vehicle. In some embodiments, provided compositions comprise about 50 wt %, about 55 wt %, about 60 wt %, about 65 wt %, about 70 wt %, about 75 wt %, about 80 wt %, about 85 wt %, about 90 wt %, or about 95 wt % vehicle, based on the total weight of the composition. In some embodiments, provided compositions comprise from about 50 wt % to about 99 wt %, about 60 wt % to about 98 wt %, about 70 wt % to about 95 wt %, about 75 wt % to about 90 wt %, or about 80 wt % to about 90 wt % vehicle, based on the total weight of the composition.

In some embodiments, the vehicle comprises one or more of a high viscosity liquid carrier material (HVLCM), a polymer (e.g., a lactic acid-based polymer), and a solvent, or any combination thereof. In some embodiments, the vehicle comprises a HVLCM. In some embodiments, the vehicle comprises a polymer (e.g., a lactic acid-based polymer). In some embodiments, the vehicle comprises a solvent. In some embodiments, the vehicle comprises a HVLCM, a polymer (e.g., a lactic acid-based polymer), and a solvent.

In some embodiments, the vehicle comprises a HVLCM, a polymer, and a solvent, wherein the relative amounts, expressed as weight ratios, are about 1: 0.1-2: 0.3-10, 1: 0.2-1: 0.4-5, 1: 0.3-0.5: 0.5-1, or 1: 0.1-0.5: 0.3-0.9, respectively.

### High Viscosity Liquid Carrier Materials (HVLCM)

In some embodiments, the present disclosure provides compositions comprising an active agent, and further comprising one or more high viscosity liquid carrier materials (HVLCMs). In some embodiments, provided compositions comprise an active agent and a vehicle comprising one or more HVLCMs. Typically, a HVLCM suitable for use in provided compositions is non-polymeric and/or not water-soluble. As used herein, the term “not water-soluble” or “non-water soluble” refers to a material that is soluble in water to a degree of less than 1% by weight under ambient conditions.

In some embodiments, the HVLCM has a viscosity of at least 5000 cP at 37° C. and does not crystallize when neat at 25° C. and at 1 atmosphere. For example, the HVLCM may have a viscosity of at least 10,000 cP, at least 15,000 cP, at least 20,000 cP, at least 25,000 cP, at least 50,000 cP, at least 100,000 cP, at least 200,000 cP, or at least 300,000 cP at 37° C.

In some embodiments, the one or more HVLCMs are selected from sucrose acetate isobutyrate, stearate esters (such as stearate esters of propylene glycol, glyceryl, diethylaminoethyl, and glycol), stearate amides or other long-chain fatty acid amides (such as N,N′-ethylene distearamide, stearamide monoethanolamine, stearamide diethanolamine, or ethylene bistearamide), cocoamine oxide, long-chain fatty alcohols (such as cetyl alcohol and stearyl alcohol), long-chain esters (such as myristyl myristate and beheny erucate), glyceryl phosphates, and acetylated sucrose distearate (i.e., Crodesta A-10). Additional materials suitable for use as the HVLCM are described in US 2004/0101557, the entire contents of which are hereby incorporated by reference.

Without wishing to be bound by any particular theory, the amount of HVLCM in provided compositions can depend on the desired properties of the composition and/or on the solvent capacity of a solvent also present in the composition. For example, if the solvent has poor solvent capacity, then the amount of solvent may be large and a corresponding reduction in the amount of HVLCM is necessary.

In some embodiments, provided compositions comprise about 5 wt %, about 10 wt %, about 25 wt %, about 30 wt %, about 40 wt %, about 50 wt %, about 55 wt %, about 60 wt %, about 70 wt %, about 80 wt %, or about 90 wt % HVLCM, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 30 wt % to about 70 wt %, or about 40 wt % to about 60 wt % HVLCM, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 25 wt % to about 65 wt %, about 30 wt % to about 70 wt %, or about 40 wt % to about 60 wt % HVLCM, based on the weight of the vehicle or the total weight of the composition.

In some embodiments, the HVLCM is sucrose acetate isobutyrate (SAIB). SAIB comprises a sucrose molecule esterified with acetic acid and isobutyric acid. SAIB is orally non-toxic and is currently used to stabilize emulsions in the food industry. It is a very viscous liquid yet undergoes dramatic changes in viscosity in the presence of heat and/or the addition of small quantities of solvent(s). For example, SAIB has a viscosity of about 2 million cP at about 25° C., of about 320,000 cP at 37° C., and of about 600 cP at 80° C. (US 2009/0087408 and U.S. Pat. No. 8,133,507, each of which is hereby incorporated by reference in its entirety). SAIB is soluble in a large number of biocompatible solvents. When in solution or in an emulsion, SAIB can be administered via injection or an aerosol spray. SAIB is compatible with cellulose esters and other polymers suitable for use in provided compositions.

In some embodiments, provided compositions comprise about 5 wt %, about 10 wt %, about 25 wt %, about 30 wt %, about 40 wt %, about 50 wt %, about 55 wt %, about 60 wt %, about 70 wt %, about 80 wt %, or about 90 wt % SAIB, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 30 wt % to about 70 wt %, or about 40 wt % to about 60 wt % SAIB, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 25 wt % to about 65 wt %, about 30 wt % to about 70 wt %, or about 40 wt % to about 60 wt % SAIB, based on the weight of the vehicle or the total weight of the composition.

Polymers

In some embodiments, the present disclosure provides compositions comprising an active agent, and further comprising one or more polymers. In some embodiments, provided compositions further comprise a vehicle comprising one or more polymers. In some embodiments, provided compositions comprise an active agent and a vehicle comprising one or more polymers. In some embodiments, the polymer is a lactic acid-based polymer, a glycolic acid-based polymer, an orthoester-based polymer, and/or a trimethylene carbonate-based polymer. In some embodiments, the polymer is a lactic acid-based polymer. Polymers that are particularly useful in provided compositions are biodegradable and/or biocompatible.

Without wishing to be bound by any particular theory, particularly useful polymers may alter the release profile of the active agent, add integrity to the composition and/or otherwise modify the properties of the composition. For example, it is desirable for provided compositions to comprise a HVLCM and a polymer which are miscible, in order to avoid phase separation of the HVLCM and the polymer. Phase separation of the HVLCM and the polymer is undesirable, because remixing may be difficult, e.g., at the time of administration, and improper mixing can affect the release profile of the active agent. Accordingly, in some embodiments, the polymer is sufficiently miscible with the HVLCM. In some embodiments, the polymer is sufficiently soluble in the composition.

In some embodiments, the polymer is or comprises a linear polymer. In some embodiments, the polymer is or comprises a branched polymer.

In some embodiments, the polymer is or comprises a saturated polymer. In some embodiments, the polymer is or comprises an unsaturated polymer.

In some embodiments, the polymer is or comprises a homopolymer. In some embodiments, the polymer is or comprises poly(lactic acid), i.e., polylactide. The terms “poly(lactic acid)” and polylactide are used interchangeably herein.

In some embodiments, the polymer is or comprises a copolymer. In some embodiments, the polymer (e.g., a lactic acid-based polymer) is or comprises a copolymer of lactic acid repeat units and another suitable repeat unit. Suitable repeat units include, but are not limited to, glycolic acid repeat units, glycolide repeat units, polyethylene glycol repeat units, caprolactone repeat units, valerolactone repeat units, trimethylene-carbonate repeat units, and the like. As used herein, “repeat unit” refers to a repetitive structural unit of a polymer. In some embodiments herein, repeat units are depicted within a set of square brackets as depicted below. It will be appreciated that each repeat unit is independent of the other, e.g., if two different monomers are used in a polymerization reaction.

In some embodiments, the polymer (e.g., a lactic acid-based polymer) is or comprises a copolymer of lactic acid repeat units and glycolic acid repeat units. Accordingly, in some embodiments, the lactic acid-based polymer is or comprises poly(lactic acid)(glycolic acid) (PLGA), i.e., poly(lactide)(glycolide). The terms “poly(lactic acid)(glycolic acid)” and “poly(lactide)(glycolide)” are used interchangeably herein.

In some embodiments, the PLGA comprises lactic acid repeat units and glycolic acid repeat units in a molar ratio of about 100:0, about 90:10, about 85:15, about 75:25, about 65:35, or about 50:50. In some embodiments, the PLGA comprises lactic acid repeat units and glycolic acid repeat units in a molar ratio of about 100:0, about 95:5, about 90:10, about 85:15, about 75:25, about 65:35, or about 50:50. In some embodiments, the PLGA comprises lactic acid repeat units and glycolic acid repeat units in a molar ratio of from about 100:0 to about 50:50, from about 100:0 to about 70:30, from about 100:0 to about 75:25, or from about 95:15 to about 85:15. In some embodiments, the PLGA comprises lactic acid repeat units and glycolic acid repeat units in a molar ratio of from about 100:0 to about 50:50, from about 100:0 to about 70:30, from about 100:0 to about 75:25, from about 95:5 to about 65:35, or from about 95:5 to about 85:15.

Without wishing to be bound by any particular theory, PLGA with a higher molar ratio of lactic acid repeat units to glycolic acid repeat units tend to be more suitable for use with SAIB and/or tend to provide longer release profiles. Accordingly, in some embodiments, the PLGA comprises lactic acid repeat units and glycolic acid repeat units in a molar ratio of greater than about 70:30, greater than about 75:25, greater than about 85:15, or greater than about 90:10.

In some embodiments, the polymer (e.g., PLGA) has a weight average molecular weight of about 4 kDa, about 8 kDa, about 10 kDa, about 12 kDa, about 14 kDa, about 16 kDa, about 18 kDa, about 20 kDa, about 30 kDa, about 40 kDa, or about 50 kDa. In some embodiments, the polymer (e.g., PLGA) has a weight average molecular weight of about 4 kDa, about 8 kDa, about 10 kDa, about 12 kDa, about 14 kDa, about 16 kDa, about 18 kDa, about 20 kDa, about 30 kDa, about 40 kDa, about 50 kDa, about 60 kDa, or about 70 kDa. In some embodiments, the polymer (e.g., PLGA) has a weight average molecular weight of from about 1 kDa to about 50 kDa, from about 4 kDa to about 40 kDa, from about 6 kDa to about 30 kDa, from about 8 kDa to about 18 kDa, from about 10 kDa to about 20 kDa, or from about 15 kDa to about 20 kDa. In some embodiments, the polymer (e.g., PLGA) has a weight average molecular weight of from about 1 kDa to about 70 kDa, from about 1 kDa to about 55 kDa, from about 1 kDa to about 50 kDa, from about 4 kDa to about 40 kDa, from about 5 kDa to about 25 kDa, from about 6 kDa to about 30 kDa, from about 8 kDa to about 18 kDa, from about 10 kDa to about 20 kDa, from about 15 kDa to about 55 kDa, or from about 15 kDa to about 20 kDa. In some embodiments, the polymer (e.g., PLGA) has a weight average molecular weight of greater than about 5 kDa, greater than about 10 kDa, greater than about 15 kDa, greater than about 16 kDa, or greater than about 18 kDa. In some embodiments, the polymer (e.g., PLGA) has a weight average molecular weight of greater than about 20 kDa, greater than about 25 kDa, greater than about 30 kDa, greater than about 40 kDa, greater than about 45 kDa, or greater than about 50 kDa.

As used herein, “weight average molecular weight” or “Mw” refers to the weighted average molecular weight of a polymer. It can be measured by any suitable means known in the art. In some embodiments, Mw is measured using gel permeation chromatography (GPC). Accordingly, in some embodiments, the polymer (e.g., PLGA) has a particular weight average molecular weight (e.g., as described herein) when measured using GPC. GPC is a column fractionation method wherein polymer molecules in solution are separated based on their size. The separated polymer molecules are detected by a detector to generate a GPC chromatogram, which is a plot of elution volume or time (related to molecular weight) versus abundance. A GPC chromatogram may be integrated to determine Mw.

In some embodiments, Mw is measured using GPC according to the following exemplary procedure: GPC samples of polymer(s) of interest are dissolved in appropriate solvent, approximately 50 mg in 10 mL of solvent. Injections of 50-200 μL are made to generate chromatograms. Chromatograms may be generated using various systems. In one embodiment, a system comprises an Agilent LC 1100 with a refractive index detector using Chemstation software. In another embodiment, a system comprises a Waters 510 pump, a Shimadzu CTO-10A column oven, and a Waters 410 differential refractometer. Data may be recorded directly to a PC via a Polymer Labs data capture unit using Caliber® software. A calibration curve may be generated using polystyrene standards. Mw, Mn, and MWD relative to polystyrene are calculated. Representative solvents for use in GPC comprise: chloroform, dichloromethane (methylene chloride), and tetrahydrofuran (THF). Representative column sets comprise: (1) a PLgel MIXED guard column in series with two Polymer Labs Mixed C columns, (2) a PLgel MIXED guard column in series with two Polymer Labs Mixed D columns, or (3) two Polymer Labs Mesopore columns in series. Representative polystyrene calibrants comprise: Polymer Labs Easical PS1 kit, Polymer Labs Easical PS2 kit, Polymer Labs S-L-10 kit.

In some embodiments, provided compositions comprise about 1 wt %, about 2 wt %, about 5 wt %, about 8 wt %, about 10 wt %, about 15 wt %, about 20 wt %, about 30 wt %, or about 40 wt % polymer (e.g., PLGA), based on the weight of the vehicle or the total weight of the composition. In some embodiments, provided compositions comprise from about 1 wt % to about 40 wt %, about 2 wt % to about 30 wt %, about 3 wt % to about 20 wt %, or about 5 wt % to about 10 wt % polymer (e.g., PLGA), based on the weight of the vehicle or the total weight of the composition. In some embodiments, provided compositions comprise from about 1 wt % to about 40 wt %, about 2 wt % to about 30 wt %, about 3 wt % to about 20 wt %, about 5 wt % to about 30 wt %, about 10 wt % to about 25 wt %, about 5 wt % to about 20 wt %, or about 5 wt % to about 10 wt % polymer (e.g., PLGA), based on the weight of the vehicle or the total weight of the composition. While not wishing to be bound by any particular theory, in some embodiments, the amount of polymer is minimized in order to minimize formation of acid and/or other byproducts upon sterilization (e.g., with gamma irradiation) and minimize acid formed in the body as the drug is released (and polymer is degrading).

Polymers described herein can be prepared using techniques that are generally known in the art. For example, polylactide can be prepared via initiation with a monoalcohol according to the following scheme:

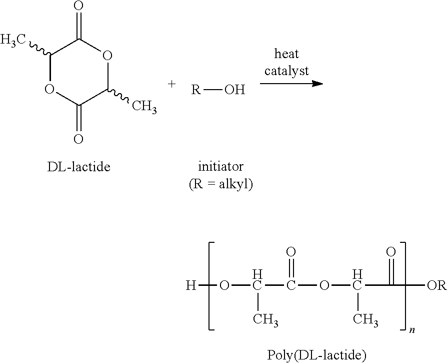

Similarly, poly(lactide)(glycolide) can be prepared via initiation with a monoalcohol according to the following scheme (of course, the arrangement of the monomers may be random as opposed to being dimeric as depicted below):

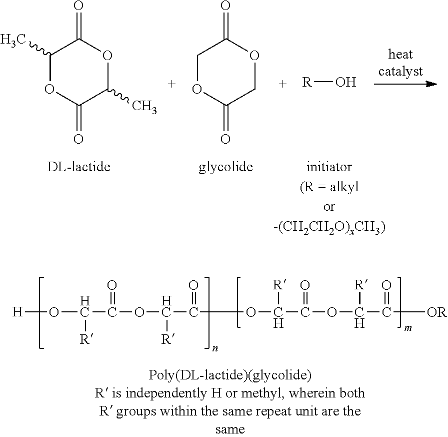

Alternatively, lactic acid-based polymers (e.g., polylactide) described herein can be prepared via initiation with a diol according to the following scheme:

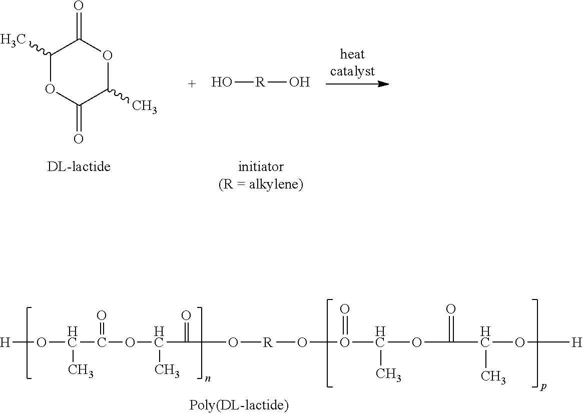

Alternatively, lactic acid-based polymers (e.g., polylactide) described herein can be prepared via initiation with water or a hydroxyl-containing carboxylic acid monomer according to the following scheme:

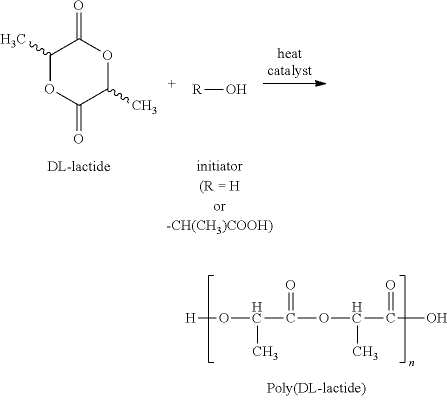

In some embodiments, the lactic acid-based polymer is prepared via initiation with an initiator selected from diols (such as 1,6-hexanediol, 1,2-propanediol, 1,3-propanediol, 1,4-butanediol, and the like), difunctionalized poly(ethyleneglycol)s (PEGs), monofunctionalized alcohols (such as 1-dodecanol, methyl lactate, ethyl lactate, and the like), monofunctional PEGs (such as methoxyPEG and the like), fatty alcohols, water, glycolic acid, lactic acid, and citric acid. In some embodiments, the initiator is a fatty alcohol or an acid. In some embodiments, the initiator is lactic acid. In some embodiments, the initiator is dodecanol (e.g., 1-dodecanol).

In some embodiments, the lactic acid-based polymer (e.g., PLGA) comprises an end group, depending on the method of preparation. In some embodiments, the end group is an alkoxy end group. In some embodiments, the alkoxy end group comprises or consists of 2 to 24 carbon atoms. In some embodiments, the alkoxy end group comprises or consists of 12 carbon atoms. In some embodiments, the end group is a hydroxy end group.

Without wishing to be bound by any particular theory, provided compositions comprising PLGA prepared via initiation with dodecanol (i.e., PLGA with an alkoxy end group comprising 12 carbon atoms) tend to exhibit desirable solubility properties. Thus, such compositions may require less solvent and/or may be more resistant to phase separation. Accordingly, in some embodiments, provided compositions comprise PLGA initiated with dodecanol (e.g., 1-dodecanol).

In some embodiments, provided compositions do not comprise cellulose acetate butyrate.

Solvent

In some embodiments, the present disclosure provides compositions comprising anactive agent, and further comprising a solvent. In some embodiments, provided compositions further comprise a solvent. In some embodiments, provided compositions further comprise a vehicle comprising a solvent. In some embodiments, provided compositions comprise an active agent and a vehicle comprising a solvent.

Without wishing to be bound by any particular theory, solvents suitable for use in provided compositions are often biocompatible, hydrophilic, water miscible, water soluble, and/or non-toxic. Suitable solvents do not cause significant tissue irritation or necrosis at the site of administration (e.g., injection or implantation) when used in conjunction with the present disclosure. Furthermore, suitable solvents are often water miscible and/or water soluble, so that they will diffuse into bodily fluids or other aqueous media. Additionally, the polymer (e.g., PLGA) and/or the HVLCM typically are soluble and/or miscible in the solvent.

In some embodiments, the solvent is or comprises an organic solvent. In some embodiments, the solvent is or comprises a polar solvent. In some embodiments, the solvent is or comprises a non-polar solvent. In some embodiments, the solvent is or comprises a hydrophilic solvent. In some embodiments, the solvent is or comprises a hydrophobic solvent.

In some embodiments, the solvent is or comprises one or more of N-methyl-pyrrolidone (NMP), dimethylsulfoxide (DMSO), propylene carbonate (PC), benzyl alcohol (BA), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one. In some embodiments, the solvent is or comprises one or more of N-methyl-pyrrolidone (NMP), dimethylsulfoxide (DMSO), propylene carbonate (PC), benzyl alcohol (BA), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one. In some embodiments, the solvent is or comprises one or more of NMP, DMSO, PC, BA, BB, ethanol, and glycofurol. In some embodiments, the solvent is or comprises one or more of NMP, DMSO, PC, BB, and ethanol.

In some embodiments, the solvent comprises propylene carbonate (PC). In some embodiments, the solvent is PC. In some embodiments, the solvent consists essentially of PC.

Without wishing to be bound by theory, the present disclosure encompasses the recognition that provided compositions that comprise a mixture of solvents may be useful for achieving certain desirable results (e.g., particular release profiles described herein). Accordingly, in some embodiments, the solvent comprises a solvent mixture (e.g., a mixture of two or more of NMP, DMSO, PC, BA, BB, dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one).

In some embodiments, the solvent is or comprises propylene carbonate (PC) and one or more solvents selected from NMP, DMSO, BA, BB, dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, and 1-dodecylazacycloheptan-2-one. In some embodiments, the solvent is or comprises propylene carbonate (PC) and dimethylsulfoxide (DMSO). In some embodiments, the solvent is or comprises propylene carbonate (PC) and ethanol.

As described above, the HVLCM is typically soluble and/or miscible in the solvent suitable for use in provided compositions. For example, SAIB is not miscible with glycerol, corn oil, peanut oil, 1,2-propanediol, polyethylene glycol (PEG200), super refined sesame oil, and super refined peanut oil. Accordingly, in some embodiments, the solvent does not comprise one or more of glycerol, corn oil, peanut oil, 1,2-propanediol, polyethylene glycol (PEG200), super refined sesame oil, and super refined peanut oil.

In some embodiments, the solvent does not comprise an alcohol. For example, in some embodiments, the solvent does not comprise ethanol. In some embodiments, the solvent does not comprise benzyl alcohol. Thus, in some embodiments, the composition is substantially free of alcohol, ethanol, and/or benzyl alcohol.

In some embodiments, the solvent does not comprise NMP.

In some embodiments, provided compositions comprise about 5 wt %, about 10 wt %, about 15 wt %, about 20 wt %, about 25 wt %, about 30 wt %, about 40 wt %, about 50 wt %, about 80 wt %, or about 90 wt % solvent, based on the weight of the vehicle or the total weight of the composition. In some embodiments, provided compositions comprise about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 80 wt %, or about 90 wt % solvent, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 10 wt % to about 40 wt %, or from about 15 wt % to about 35 wt % solvent, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 20 wt % to about 60 wt %, from about 25 wt % to about 55 wt %, from about 10 wt % to about 40 wt %, or from about 15 wt % to about 35 wt % solvent, based on the weight of the vehicle or the total weight of the composition.

In some embodiments, provided compositions comprise about 5 wt %, about 10 wt %, about 15 wt %, about 20 wt %, about 25 wt %, about 30 wt %, about 40 wt %, about 50 wt %, about 80 wt %, or about 90 wt % PC, based on the weight of the vehicle or the total weight of the composition. In some embodiments, provided compositions comprise about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 80 wt %, or about 90 wt % PC, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 10 wt % to about 40 wt %, or from about 15 wt % to about 35 wt % PC, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 20 wt % to about 60 wt %, from about 25 wt % to about 55 wt %, from about 10 wt % to about 40 wt %, or from about 15 wt % to about 35 wt % PC, based on the weight of the vehicle or the total weight of the composition.

Other Components:

In some embodiments, provided compositions optionally further comprise one or more additional components (i.e., additives) in order to modify the properties of the compositions as desired. The additives may be present in any amount that is sufficient to impart the desired properties. The amount of additive used will generally be a function of the nature of the additive and the effect to be achieved, and can be easily determined by one of skill in the art. For example, when present, additive(s) are typically present in provided compositions from about 0.1 wt % to about 20 wt %, based on the weight of the vehicle or the total weight of the composition.

In some embodiments, provided compositions further comprise a buffer, in order to, e.g., modify the pH of the composition.

In some embodiments, provided compositions further comprise one or more additional polymers (i.e., a polymer other than the lactic acid-based polymer), such as a non-biodegradable polymer. Non-limiting examples of such polymers include polyacrylates, ethylene-vinyl acetate polymers, cellulose and cellulose derivatives, acyl substituted cellulose acetates and derivatives thereof (such as cellulose acetate butyrate and cellulose acetate propionate), non-erodible polyurethanes, polystyrenes, polyvinyl chloride, polyvinyl fluoride, poly(vinyl imidazole), chlorosulphonated polyolefins, polyethylene oxide, polyethylene, polyvinyl pyrrolidone, ethylene vinylacetate, and polyethylene glycol.

In some embodiments, provided compositions further comprise one or more a natural or synthetic oils and/or fats in order to, e.g., increase the hydrophobicity of provided compositions and thereby slowing degradation and/or water uptake of the composition. Exemplary suitable natural and synthetic oils include vegetable oil, peanut oil, medium chain triglycerides, almond oil, olive oil, sesame oil, peanut oil, fennel oil, camellia oil, corn oil, castor oil, cotton seed oil, soybean oil, either crude or refined, and medium chain fatty acid triglycerides. Exemplary suitable fats include lard and tallow.

In some embodiments, provided compositions further comprise one or more carbohydrates and/or carbohydrate derivatives. Non-limiting examples of carbohydrates and carbohydrate derivatives include monosaccharides (e.g., simple sugars such as fructose and glucose), disaccharides (such as sucrose, maltose, cellobiose, and lactose), and polysaccharides.

In some embodiments, provided compositions further comprise one or more preservatives (such as paraben derivatives, e.g., methyl paraben and propyl paraben), stabilizers, anti-oxidants (such as butyl hydroxyanisole, butyl hydroxytoluene, propyl gallate, vitamin E acetate, and purified hydroquinone), coloring agents, isotonic agents, humectants (such as sorbitol), sequesterants (such as citric acid), vitamins, vitamin precursors, and/or surfactants.

In some embodiments, provided compositions further comprise one or more viscosity enhancers, antioxidants, preservatives, and particle stabilizers. For instance, provided compositions may comprise one or more of ricinoleic acid, polyoxyethylene-polyoxypropylene block copolymer, polyvinylpyrrolidone, polyethyeleneglycol (e.g., PEG4000), and Cremophor EL® ethoxylated castor oil which includes polyethylene glycol ether.

The present disclosure also encompasses the recognition that it may be desirable to control or reduce water content in provided formulations. For example, if the presence of water increases the rate of polymer and/or active agent degradation, removing and/or minimizing the amount of water may be desirable. Accordingly, the present disclosure also provides compositions having surprisingly low water content. In some embodiments, provided compositions are substantially free of water. In some embodiments, provided compositions comprise less than about 0.5 wt %, less than about 0.35 wt %, less than about 0.25 wt %, less than about 0.2 wt %, less than about 0.15 wt %, less than about 0.1%, less than about 0.01 wt % or less than about 0.005 wt % water, based on the weight of the vehicle or the total weight of the composition. In some embodiments, provided compositions comprise from about 0.001 wt % to about 0.35 wt %, from about 0.001 wt % to about 0.25 wt %, from about 0.001 wt % to about 0.1 wt %, from about 0.001 wt % to about 0.01 wt %, or from about 0.001 wt % to about 0.005 wt % water, based on the weight of the vehicle or the total weight of the composition.

### Provided Compositions:

In some embodiments, provided compositions have a total weight of from about 25 mg to about 10,000 mg, from about 50 mg to about 5000 mg, from about 100 mg to about 4000 mg, from about 150 mg to about 3000 mg, or from about 200 mg to about 2000 mg. In some embodiments, provided compositions have a total weight of about 50 mg, about 100 mg, about 200 mg, about 300 mg, about 400 mg, about 500 mg, about 600 mg, about 700 mg, about 800 mg, about 900 mg, about 1000 mg, about 1500 mg, about 2000 mg, about 2500 mg, about 3000 mg, about 3500 mg, about 4000 mg, about 4500 mg, or about 5000 mg.

In some embodiments, provided compositions have a total volume of from about 0.025 mL to about 10 mL, from about 0.05 mL to about 5 mL, from about 0.1 mL to about 4 mL, from about 0.15 mL to about 3 mL, or from about 0.2 mL to about 2 mL. In some embodiments, provided compositions have a total volume of about 0.05 mL, about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.5 mL, or about 2 mL.

In some embodiments, the present disclosure provides a composition comprising:
(i) from about 5 wt % to about 35 wt % active agent, based on the weight of the vehicle or the total weight of the composition;
(ii) from about 5 wt % to about 20 wt % poly(lactic acid)(glycolic acid), based on the weight of the vehicle or the total weight of the composition;
(iii) from about 10 wt % to about 40 wt % propylene carbonate, based on the weight of the vehicle or the total weight of the composition; and
(iv) from about 40 wt % to about 60 wt % sucrose acetate isobutyrate, based on the weight of the vehicle or the total weight of the composition.

In some embodiments, the present disclosure provides a composition comprising:
(i) from about 5 wt % to about 15 wt % active agent, based on the weight of the vehicle or the total weight of the composition;
(ii) from about 5 wt % to about 10 wt % poly(lactic acid)(glycolic acid), based on the weight of the vehicle or the total weight of the composition;
(iii) from about 20 wt % to about 30 wt % propylene carbonate, based on the weight of the vehicle or the total weight of the composition; and
(iv) from about 50 wt % to about 60 wt % sucrose acetate isobutyrate, based on the weight of the vehicle or the total weight of the composition.

In some embodiments, the present disclosure provides a composition comprising:
(i) from about 5 wt % to about 35 wt % active agent, based on the weight of the vehicle or the total weight of the composition;
(ii) from about 5 wt % to about 30 wt % poly(lactic acid)(glycolic acid), based on the weight of the vehicle or the total weight of the composition;
(iii) from about 20 wt % to about 60 wt % propylene carbonate, based on the weight of the vehicle or the total weight of the composition; and
(iv) from about 25 wt % to about 65 wt % sucrose acetate isobutyrate, based on the weight of the vehicle or the total weight of the composition.

In some embodiments, the present disclosure provides a composition comprising,
based on the total weight of the composition:
(i) about 11.1 wt % active agent;
(ii) about 8.9 wt % poly(lactic acid)(glycolic acid);
(iii) about 24.9 wt % propylene carbonate; and
(iv) about 55.1 wt % sucrose acetate isobutyrate.

In some embodiments, the present disclosure provides a composition comprising:
(i) about 11.1 wt % active agent, based on the total weight of the composition;
(ii) about 10 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 28 wt % propylene carbonate, based on the weight of the vehicle; and
(iv) about 62 wt % sucrose acetate isobutyrate, based on the weight of the vehicle.

In some embodiments, the present disclosure provides a composition comprising:
(i) about 11.1 wt % active agent, based on the total weight of the composition;
(ii) about 19 wt % poly(lactic acid)(glycolic acid) (e.g., PLGA with a weight average molecular weight of about 8 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 37 wt % propylene carbonate, based on the weight of the vehicle; and
(iv) about 44 wt % sucrose acetate isobutyrate, based on the weight of the vehicle.

In some embodiments, the present disclosure provides a composition comprising:
(i) about 22.2 wt % active agent, based on the total weight of the composition;
(ii) about 19 wt % poly(lactic acid)(glycolic acid) (e.g., PLGA with a weight average molecular weight of about 8 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 37 wt % propylene carbonate, based on the weight of the vehicle; and
(iv) about 44 wt % sucrose acetate isobutyrate, based on the weight of the vehicle.

In some embodiments, the present disclosure provides a composition comprising:
(i) about 11.1 wt % active agent, based on the total weight of the composition;
(ii) about 20 wt % poly(lactic acid)(glycolic acid) (e.g., PLGA with a weight average molecular weight of about 48 kDa, e.g., when measured using GPC, and/or with lactic acid repeat units and glycolic acid repeat units in a molar ratio of about 65:35 and/or initiated with 1-dodecanol), based on the weight of the vehicle;
(iii) about 55 wt % propylene carbonate, based on the weight of the vehicle; and
(iv) about 25 wt % sucrose acetate isobutyrate, based on the weight of the vehicle.

In some embodiments, the present disclosure provides a composition comprising:
(i) about 11.1 wt % active agent, based on the total weight of the composition;
(ii) about 25 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 37 wt % propylene carbonate, based on the weight of the vehicle; and
(iv) about 38 wt % sucrose acetate isobutyrate, based on the weight of the vehicle.

In some embodiments, the present disclosure provides a composition comprising:
(i) about 11.1 wt % active agent, based on the total weight of the composition;
(ii) about 20 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 9 wt % dimethylsulfoxide, based on the weight of the vehicle;
(iv) about 21 wt % propylene carbonate, based on the weight of the vehicle; and
(v) about 50 wt % sucrose acetate isobutyrate, based on the weight of the vehicle.

In some embodiments, the present disclosure provides a composition comprising:
(i) about 11.1 wt % active agent, based on the total weight of the composition;
(ii) about 20 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 5.5 wt % ethanol, based on the weight of the vehicle;
(iv) about 21.5 wt % propylene carbonate, based on the weight of the vehicle; and
(v) about 53 wt % sucrose acetate isobutyrate, based on the weight of the vehicle.

In some embodiments, the present disclosure provides a composition comprising:
(i) about 11.1 wt % active agent, based on the total weight of the composition;
(ii) about 20 wt % poly(lactic acid)(glycolic acid) (e.g., PLGA with a weight average molecular weight of about 40 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 46 wt % propylene carbonate, based on the weight of the vehicle; and
(iv) about 34 wt % sucrose acetate isobutyrate, based on the weight of the vehicle.

In some embodiments, the present disclosure provides a composition comprising:
(i) about 11.1 wt % active agent, based on the total weight of the composition;
(ii) about 20 wt % poly(lactic acid)(glycolic acid) (e.g., PLGA with a weight average molecular weight of about 51 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 55 wt % propylene carbonate, based on the weight of the vehicle; and
(iv) about 25 wt % sucrose acetate isobutyrate, based on the weight of the vehicle.

In some embodiments, the present disclosure provides a composition comprising:
(i) about 11.1 wt % active agent, based on the total weight of the composition;
(ii) about 20 wt % poly(lactic acid)(glycolic acid) (e.g., PLGA with a weight average molecular weight of about 29 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 43 wt % propylene carbonate, based on the weight of the vehicle; and
(iv) about 37 wt % sucrose acetate isobutyrate, based on the weight of the vehicle.

In some embodiments, the present disclosure provides a composition comprising:
(i) from about 1 mg to about 500 mg active agent;
(ii) from about 1 mg to about 500 mg poly(lactic acid)(glycolic acid);
(iii) from about 5 mg to about 1000 mg propylene carbonate; and
(iv) from about 10 mg to about 2000 mg sucrose acetate isobutyrate.

In some embodiments, the present disclosure provides a composition of Table 1A, Table 1B, Table 3A, Table 3B, Table 16, Table 19, or Table 24 below.

### Characteristics of Provided Compositions

As described above, the present disclosure provides novel long-acting formulations of an active agent. Provided compositions achieve certain desirable characteristics, as described herein.

For example, without wishing to be bound by any particular theory, compositions (e.g., formulations and/or vehicles provided herein) that are monophasic are easy to store, are stable, and/or enable consistent administration of the active agent. Monophasic compositions are particularly desirable to avoid inconsistencies related to administration of the composition. For instance, if a composition requires re-mixing because phase separation occurs after storage for a period of time, some subjects may not receive the same amount of each component of a provided composition, which may result in suboptimal outcomes, e.g., release profiles. Accordingly, in some embodiments, provided formulations are monophasic. In some embodiments, provided formulations comprise suitable amounts of each component (e.g., an active agent, HVLCM, polymer, and/or solvent), so that the formulation is monophasic. In some embodiments, provided vehicles are monophasic. In some embodiments, provided vehicles comprise suitable amounts of each component (e.g., HVLCM, polymer, and/or solvent), so that the vehicle is monophasic.

Phase separation may be investigated by visual techniques well known to those skilled in the art. Some compositions may be rendered into a uniform clear solution by sufficient heating and mixing. Yet, when cooled to room temperature, two clear liquid phases may form. Sometimes, two clear layers may not be easy to detect, thus requiring strong light and thorough inspection to discern the boundary between the two phases. In some cases, compositions may appear clear and uniform on initial cooling to room temperature, but when left quiescent at room temperature for a period of time, the compositions may separate into two phases. In some cases, the composition may turn cloudy and slowly separate into two phases.

In some embodiments, provided formulations remain monophasic over a period of time (i.e., no phase separation of provided formulations is observed over a period of time). In some embodiments, provided formulations are monophasic after storage for 1 week, 2 weeks, 1 month, 2 months, 6 months, or longer. In some embodiments, provided formulations are monophasic after storage at 0° C., 10° C., 25° C., 37° C., or cooler, or warmer. In some embodiments, provided formulations are monophasic after storage at 0° C., 10° C., 25° C., 37° C., or cooler, or warmer for 1 week, 2 weeks, 1 month, 2 months, 6 months, or longer.

In some embodiments, provided vehicles remain monophasic over a period of time (i.e., no phase separation of provided vehicles is observed over a period of time). In some embodiments, provided vehicles are monophasic after storage for 1 week, 2 weeks, 1 month, 2 months, 6 months, or longer. In some embodiments, provided vehicles are monophasic after storage at −20° C., −10° C., 0° C., 10° C., 25° C., 37° C., or cooler, or warmer. In some embodiments, provided vehicles are monophasic after storage at −20° C., −10° C., 0° C., 10° C., 25° C., 37° C., or cooler, or warmer for 1 week, 2 weeks, 1 month, 2 months, 6 months, or longer.

In some instances, provided formulations comprising an active agent are not monophasic (e.g., are suspensions). Without wishing to be bound by any particular theory, formulations in which the active agent is not fully soluble in the vehicle may also be useful as long-acting formulations. In fact, such suspensions may enable even slower release of the active agent, which upon administration will need to dissolve first before releasing into the body. Accordingly, in some embodiments, provided compositions are suspensions. In some embodiments, provided compositions are suspensions of the active agent in the vehicle. In some embodiments, provided compositions are suspensions of the active agent in the vehicle, wherein the vehicle is monophasic.

Without wishing to be bound by any particular theory, a viscosity of provided compositions is within a desirable range, in order to, e.g., be easily administered through a needle or other suitable means for administration while still achieving desirable long-acting release characteristics. Accordingly, in some embodiments, provided compositions have a viscosity of less than about 20,000 cP, less than about 10,000 cP, less than about 8,000 cP, less than about 6,000 cP, less than about 4,000 cP, or less than about 2,000 cP at a shear rate of 500 s⁻¹ at 25° C. In some embodiments, provided compositions have a viscosity of less than about 10,000 cP, less than about 8,000 cP, less than about 6,000 cP, less than about 4,000 cP, or less than about 2,000 cP at a shear rate of 500 s⁻¹ at 25° C. In some embodiments, provided compositions have a viscosity from about 50 cP to about 10,000 cP, from about 500 cP to about 8,000 cP, from about 500 cP to about 6,000 cP, or from about 1,000 cP to about 10,000 cP at a shear rate of 500 s⁻¹ at 25° C. In some embodiments, provided compositions have a viscosity from about 10 cP to about 20,000 cP, from about 50 cP to about 10,000 cP, from about 500 cP to about 8,000 cP, from about 500 cP to about 6,000 cP, or from about 1,000 cP to about 10,000 cP at a shear rate of 500 s⁻¹ at 25° C.

Vehicle viscosity is related to the viscosity of provided formulations. In some embodiments, provided vehicles have a viscosity of less than about 10,000 cP, less than about 8,000 cP, less than about 6,000 cP, less than about 4,000 cP, or less than about 2,000 cP at a shear rate of 500 s⁻¹ at 25° C. In some embodiments, provided vehicles have a viscosity from about 50 cP to about 10,000 cP, from about 100 cP to about 8,000 cP, from about 200 cP to about 6,000 cP, or from about 500 cP to about 2,000 cP at a shear rate of 500 s⁻¹ at 25° C. In some embodiments, provided vehicles have a viscosity from about 10 cP to about 10,000 cP, from about 50 cP to about 10,000 cP, from about 100 cP to about 8,000 cP, from about 200 cP to about 6,000 cP, or from about 500 cP to about 2,000 cP at a shear rate of 500 s⁻¹ at 25° C.

In some embodiments, provided compositions are surprisingly shear-thinning (i.e., provided compositions have lower viscosities at higher shear, compared to viscosity at lower shear or no shear).

Additionally or alternatively, provided compositions can be injected within a suitable amount of time (e.g., within seconds) under a suitable amount of force, which is desirable, e.g., for convenient administration. For example, in some embodiments, provided compositions can be injected in less than about 20 seconds, less than about 15 seconds, less than about 13 seconds, less than about 10 seconds, or less than about 8 seconds with 5 lbf. In some embodiments, provided compositions can be injected in less than about 60 seconds, less than about 50 seconds, less than about 40 seconds, less than about 30 seconds, less than about 20 seconds, less than about 15 seconds, less than about 13 seconds, less than about 10 seconds, or less than about 8 seconds with 5 lbf. In some embodiments, provided compositions can be injected within from about 1 second to about 30 seconds, from about 2 seconds to about 20 seconds, from about 4 seconds to about 15 seconds, from about 6 seconds to about 12 seconds, or from about 6 seconds to about 10 seconds with 5 lbf. In some embodiments, provided compositions can be injected within from about 1 second to about 60 seconds, from about 1 second to about 30 seconds, from about 2 seconds to about 20 seconds, from about 4 seconds to about 15 seconds, from about 6 seconds to about 12 seconds, or from about 6 seconds to about 10 seconds with 5 lbf. In some embodiments, provided compositions can be injected within from about 1 second to about 30 seconds, from about 2 seconds to 20 seconds, from about 4 seconds to about 15 seconds, or from about 6 seconds to about 10 seconds with 10 lbf. In some embodiments, provided compositions can be injected within from about 1 second to about 60 seconds, from about 1 second to about 30 seconds, from about 2 seconds to 20 seconds, from about 4 seconds to about 15 seconds, or from about 6 seconds to about 10 seconds with 10 lbf.

Furthermore, it is desirable for provided compositions to be stable to storage for a period of time. In some embodiments, provided compositions comprise at least about 90%, about 95%, about 97%, about 98%, about 99%, or greater of the active agent (relative to the initial amount of active agent) after storage for 1 week, 2 weeks, 1 month, 2 months, 6 months, or longer at 0° C., 10° C., 25° C., 37° C., or cooler, or warmer. In some embodiments, provided compositions comprise no more than about 10%, about 5%, about 3%, about 2%, about 1%, or less of total degradation products (relative to the initial amount of total degradation products) after storage for 1 week, 2 weeks, 1 month, 2 months, 6 months, or longer at 0° C., 10° C., 25° C., 37° C., or cooler, or warmer.

As described above, provided compositions are useful as long-acting formulations. Accordingly, in some embodiments, upon administration of a provided composition to a subject or across a population of subjects, a therapeutically effective concentration of active agent is maintained (i.e., concentration of active agent is above a minimum threshold concentration (Cmin)) for a sufficient period of time.

In some embodiments, when administered subcutaneously as a single dose to a subject, provided compositions achieve a plasma active agent concentration of greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer. In some embodiments, when administered subcutaneously as a single dose, provided compositions achieve a plasma active agent concentration of greater than about 0.01 ng/mL, about 0.1 ng/mL, or about 0.5 ng/mL for about 10 days to about 75 days, about 20 days to about 70 days, about 20 days to about 40 days, about 25 days to about 35 days, about 30 days to about 65 days, about 40 days to about 60 days, or about 50 days to about 60 days.

In some embodiments, when administered subcutaneously as a single dose to a subject, provided compositions achieve a therapeutically effective plasma concentration of the active agent, or a metabolite thereof, for at least about 7 days, about 14 days, about 21 days, about 28 days, or more.

In some embodiments, upon administration, provided compositions achieve a slower release of an active agent when compared to a reference composition. In some such embodiments, the reference composition is a tablet comprising 25 mg active agent administered once daily.

In some embodiments, upon administration, provided compositions achieve a therapeutically effective concentration of an active agent comparable to that of a reference composition. In some such embodiments, the reference composition is a tablet comprising 25 mg active agent administered once daily.

In some embodiments, when a provided composition is placed in phosphate-buffered saline at 37° C. (e.g., at pH 6.0 or 7.4), the amount of an active agent released from the provided composition after 4 weeks is about 20%, about 30%, about 40%, about 50%, about 60%, about 70%, about 80%, about 90%, or about 100% of the total amount of the active agent in the provided composition. In some embodiments, when a provided composition is placed in either (1) phosphate-buffered saline at 37° C. (e.g., at pH 6.0 or 7.4) for 4 weeks or (2) phosphate-buffered saline at 37° C. (e.g., at pH 6.0 or 7.4) for 10 days and then 20 mM KH₂PO₄, pH 6.0, with 0.9% NaCl buffer for 18 days, the amount of an active agent released from the provided composition is from about 20% to about 100%, about 20% to about 80%, about 40% to about 100%, about 50% to about 100%, or about 40% to about 80% of the total amount of the active agent in the provided composition.

In some embodiments, when a provided composition is placed in phosphate-buffered saline at 37° C. (e.g., at pH 6.0 or 7.4), the amount of an active agent released from the provided composition after 24 hours is less than about 40%, less than about 30%, less than about 20%, or less than about 10% of the amount of active agent released after 28 days. In some embodiments, when a provided composition is placed in phosphate-buffered saline at 37° C. (e.g., at pH 6.0 or 7.4), the amount of active agent released from the provided composition after 24 hours is less than about 50%, less than about 40%, less than about 30%, less than about 20%, or less than about 10% of the amount released after 28 days.

In some embodiments, when a provided composition is placed in phosphate-buffered saline at 37° C. (e.g., at pH 6.0 or 7.4), the amount of an active agent released from the provided composition after 28 days is greater than about 30%, greater than about 40%, greater than 50%, greater than about 60%, greater than about 70%, or greater than about 80% of a total amount of the active agent in the composition. In some embodiments, when a provided composition is placed in phosphate-buffered saline at 37° C. (e.g., at pH 6.0 or 7.4), the amount of active agent released from the provided composition after 28 days is greater than about 20%, greater than about 30%, greater than about 40%, greater than 50%, greater than about 60%, greater than about 70%, or greater than about 80% of a total amount of active agent in the composition.

### Methods of Preparing Provided Compositions

The present disclosure also provides method of manufacturing provided compositions. In some embodiments, a method of manufacturing a provided composition comprises:
(i) providing a vehicle comprising a high viscosity liquid carrier material (HVLCM); and
(ii) combining the vehicle with an active agent under suitable conditions to give the provided composition.

In some embodiments, the method comprises combining the vehicle with an active agent under suitable conditions, wherein the suitable conditions comprise combining the vehicle with the active agent with stirring (e.g., stirring with a stir bar, an overhead stirrer, or a homogenizer). In some embodiments, the active agent is added to the vehicle (e.g., in a controlled manner). In some embodiments, the method further comprises homogenizing the mixture of the active agent and vehicle (e.g., in order to obtain a uniform dispersion). In some embodiments, the active agent is provided and/or utilized in crystalline form.

In some embodiments, the vehicle comprises a HVLCM, a polymer, and a solvent. Accordingly, in some embodiments, the method further comprises mixing the HVLCM, the polymer, and the solvent under suitable conditions. In some such embodiments, suitable conditions comprises a suitable temperature of about 25° C., about 30° C., about 40° C., about 50° C., about 60° C., or about 70° C., or any range therein. In some such embodiments, suitable conditions comprise a suitable period of time of about 30 min, about 1 h, about 2 h, about 3 h, about 4 h, about 8 hr, about 16 h, about 24 h, or about 48 h, or any range therein. In some such embodiments, suitable conditions comprise a suitable mixing speed of about 5 rpm, about 10 rpm, about 20 rpm, about 25 rpm, or about 30 rpm.

In some embodiments, the method further comprises mixing the polymer and the solvent before combining with the HVLCM. In some embodiments, the method further comprises mixing the polymer and the solvent under suitable conditions. In some such embodiments, suitable conditions comprise a suitable temperature of about 10° C., about 20° C., about 25° C., about 30° C., about 40° C., or about 50° C., or any range therein. In some such embodiments, suitable conditions comprise a suitable period of time of about 30 min, about 1 h, about 2 h, about 3 h, about 4 h, about 8 h, about 12 h, about 16 h, or about 24 h, or any range therein. In some such embodiments, suitable conditions comprise a suitable mixing speed of about 5 rpm, about 10 rpm, about 20 rpm, about 25 rpm, or about 30 rpm. In some embodiments, the method further comprises allowing the polymer to warm to room temperature before combining with the solvent.

In some embodiments, the method further comprises heating the HVLCM before combining with the polymer and the solvent. In some such embodiments, the HVLCM is heated to about 50° C., about 60° C., about 70° C., about 80° C., about 90° C., or about 100° C., or any range therein.

In some embodiments, the method further comprises allowing the vehicle to cool to room temperature before combining with the active agent.

In some embodiments, the active agent is milled before combining with the vehicle. In some embodiments, the active agent is dissolved in the composition. In some embodiments, the active agent is suspended in the composition. In some embodiments, the active agent has a median particle size, as measured by laser diffraction, from about 0.1 μm to about 100 μm, about 0.2 μm to about 90 μm, about 0.25 μm to about 80 μm, about 0.5 μm to about 70 μm, about 1 μm to about 70 μm, about 2 μm to about 60 μm, about 5 μm to about 60 μm, about 10 μm to about 50 μm, or about 10 μm to about 40 μm.

In some embodiments, the method further comprises removing water so that the provided composition comprises less than about 0.5 wt %, less than about 0.35 wt %, less than about 0.25 wt %, less than about 0.2 wt %, less than about 0.15 wt %, less than about 0.1%, less than about 0.01 wt % or less than about 0.005 wt % water, based on the weight of the vehicle or the total weight of the composition. In some embodiments, the method further comprises removing water so that the provided composition comprises from about 0.001 wt % to about 0.35 wt %, from about 0.001 wt % to about 0.25 wt %, from about 0.001 wt % to about 0.1 wt %, from about 0.001 wt % to about 0.01 wt %, or from about 0.001 wt % to about 0.005 wt % water based on the weight of the vehicle or the total weight of the composition. In some embodiments, the method further comprises removing the water under an inert gas (e.g., nitrogen). In some embodiments, the method further comprises removing the water by heating and/or mixing the mixture.

In some embodiments, the method further comprises sterilizing the provided composition. In some embodiments, the method further comprises sterilizing the provided composition with gamma irradiation. In some embodiments, the gamma irradiation dose is less than about 25 kGy, less than about 20 kGy, less than about 15 kGy, or less than about 10 kGy. In some embodiments, the gamma irradiation dose is from about 10 kGy to about 25 kGy, about 15 kGy to about 25 kGy, about 15 kGy to about 20 kGy, or about 20 kGy to about 25 kGy.

In some embodiments, the method comprises irradiating the active agent before combining the active agent with the vehicle. In some embodiments, the method comprises filter sterilizing the vehicle before combining the vehicle with the active agent. In some embodiments, the method comprises combining the active agent (e.g., the active agent that has been irradiated) with the vehicle (e.g., the vehicle that has been filter sterilized) under aseptic conditions.

In some embodiments, the provided composition comprises at least 95%, at least 97%, at least 98%, at least 99%, or more of the active agent after gamma irradiation, relative to the initial amount of the active agent before gamma irradiation. In some embodiments, the provided composition comprises less than about 5%, less than about 3%, less than about 2%, less than about 1%, or less of total degradation products after gamma irradiation. In some embodiments, the provided composition comprises no more than about 5%, no more than about 3%, no more than about 2%, no more than about 1% or less of additional degradation products after gamma irradiation, relative to the initial amount of total degradation products before gamma irradiation.

### Dosing and Administration

The present disclosure also provides compositions comprising an active agent in various forms for administration, which are useful in the methods described herein.

In some embodiments, provided compositions are formulated for subcutaneous, intramuscular, or parenteral administration. Accordingly, in some embodiments, provided methods comprise administering a provided composition subcutaneously, intramuscularly, or parenterally.

In some embodiments, administration of provided compositions is accomplished with a syringe and needle, pump, patch-pump, bolus injector, infusion, auto-injector, needle-free injector, or the like. Accordingly, the present disclosure also provides a receptacle containing a provided composition. In some embodiments, the receptacle is a syringe, pump, patch-pump, bolus injector, infusion, auto-injector, or needle-free injector.

In some embodiments, administration of provided compositions is accomplished via a syringe and needle. Accordingly, in some embodiments, the present disclosure provides a syringe comprising a provided composition. In some embodiments, the syringe is equipped with a needle. In some such embodiments, the needle has a length of <1 inch, <0.625 inches, or <0.5 inches. In some embodiments, the needle has a gauge ranging from 18 G to 26 G, such as 19 G to 25 G, 20 G to 24 G, or 21 G to 23 G. In some embodiments, the needle has a gauge ranging from 16 G to 26 G or from 18 G to 26 G, such as 19 G to 25 G, 20 G to 24 G, or 21 G to 23 G.

In some embodiments, administration of provided compositions is accomplished via a pre-filled syringe or an auto-injector. Accordingly, in some embodiments, the present disclosure provides a pre-filled syringe or an auto-injector comprising a provided composition.

In some embodiments, administration of provided compositions is accomplished via a needle-free injector. Accordingly, in some embodiments, the present disclosure provides a needle-free injector comprising a provided composition.

The present disclosure also provides a vial containing a provided composition.

In some embodiments, provided compositions are administered by a health care professional. In some embodiments, provided compositions are administered by a non-health care professional. In some embodiments, provided compositions are self-administered.

In some embodiments, the present disclosure provides a dosage form comprising an active agent. In some embodiments, the dosage form further comprises sucrose acetate isobutyrate, a lactic acid-based polymer, and/or a solvent, according to compositions described herein.

In some embodiments, the dosage form is a liquid dosage form. In some embodiments, the liquid dosage form is provided as a solution or a suspension.

In some embodiments, the dosage form is provided in a receptacle selected from a syringe, pump, patch-pump, bolus injector, infusion, auto-injector, or needle-free injector.

The present disclosure also provides dosing regimens for administering provided compositions that are useful in the methods described herein.

Without wishing to be bound by any particular theory, long-acting formulations (as provided herein) allow for less frequent dosing, which can, e.g., increase patient compliance with a dosing regimen. Provided compositions may be particularly useful in patient populations that are prone to non-compliance (e.g., patients who are taking multiple drugs a day and/or who are taking drugs multiple times a day). Additionally or alternatively, provided compositions may be particularly useful for treating and/or preventing diseases or disorders, wherein compliance to a rigid therapeutic regimen is especially beneficial (e.g., combination therapy which relies on the action of multiple agents together). Therefore, in some embodiments, the present disclosure provides methods of increasing subject compliance with a therapeutic regimen comprising an active agent.

As described above, provided compositions are long-acting formulations and therefore allow for less frequent dosing than other dosage forms of active agents. Accordingly, in some embodiments, provided compositions are administered once a day, once a week, twice a month, once a month, once every two months, or once every three months.

Under some circumstances, it may be beneficial to administer a loading dose of an active agent prior to and/or concurrently with provided long-acting formulations, in order to, e.g., achieve a suitable release profile. As used herein, a “loading dose” is one or more doses of an active agent administered in addition to a long-acting formulation. A loading dose may be used to compensate for inadequate plasma levels of the active agent, while a steady state concentration is reached from the long-acting formulation. In some embodiments, methods of administering a provided composition, further comprise administering a loading dose of an active agent, which may be the same or different as the active agent in the provided composition. In some such embodiments, the loading dose is administered prior to and/or concurrently with administering a provided composition. In some such embodiments, the loading dose is administered orally or by injection.

In some embodiments, methods of administering a provided composition do not further comprise administering a loading dose.

### EXEMPLARY EMBODIMENTS

The following numbered aspects, while non-limiting, are exemplary of certain aspects of the present disclosure:

1 A composition comprising:
a non-polymeric, non-water soluble high viscosity liquid carrier material (HVLCM) having a viscosity of at least 5000 cP at 37° C. that does not crystallize neat at 25° C. and 1 atmosphere;
a lactic acid-based polymer;
a solvent; and
0.001 wt % to 0.35 wt %, based on total weight of the composition, of water.
   2. The composition of aspect 1, further comprising an active agent, wherein the active agent optionally:

has a solubility in the composition at 25° C. of less than about 10 mg/ml;

comprises at least one member selected from peptide, protein, antibody, carbohydrate, small molecule having a molecular weight less than 1500 Daltons, nucleic acid, and nucleoside; and/or

comprises particles having a median particle size, as measured by laser diffraction, ranging from 0.5 micrometers to 100 micrometers.

3. A composition comprising:
(i) an active agent having at least one pKa less than 9; and
(ii) a vehicle comprising a non-polymeric, non-water soluble high viscosity liquid carrier material (HVLCM) having a viscosity of at least 5000 cP at 37° C. that does not crystallize neat at 25° C. and 1 atmosphere.
   4. The composition of any one of aspects 1-3, wherein the HVLCM is or comprises at least one member selected from sucrose acetate isobutyrate, a stearate ester, propylene glycol, glyceryl, diethylaminoethyl, glycol, a stearate amide, a long-chain fatty acid amide, N,N′-ethylene distearamide, stearamide monoethanolamine (MEA), stearamide diethanolamine (DEA), ethylene bistearamide, cocoamine oxide, a long-chain fatty alcohol, cetyl alcohol, stearyl alcohol, long-chain ester, myristyl myristate, beheny erucate, a glyceryl phosphate, and acetylated sucrose distearate.
   5. The composition of any one of aspects 1-4, wherein the HVLCM is or comprises sucrose acetate isobutyrate.
   6. The composition of any one of aspects 2-5, wherein the active agent comprises particles having a median particle size, as measured by laser diffraction, ranging from 0.5 micrometers to 100 micrometers.
   7. The composition of any one of aspects 2-6, wherein the active agent comprises a small molecule having a molecular weight less than 1500 Daltons.
   8. The composition of any one of aspects 2-7, wherein the active agent has a water solubility of less than or equal to 1 mg/mL.
   9. The composition of any one of aspects 2-8, wherein the active agent has at least one pKa less than 8.
   10. The composition of any one of aspects 2-9, wherein the active agent has at least one pKa less than 7.
   11. The composition of any one aspects 2-10, wherein the composition comprises from about 1 wt % to about 50 wt %, about 2 wt % to about 40 wt %, about 5 wt % to about 30 wt %, about 10 wt % to about 25 wt %, or about 10 wt % to about 20 wt % active agent, based on weight of the vehicle or weight of the composition.
   12. The composition of any one of the preceding aspects, wherein the composition comprises from about 5 wt % to about 95 wt %, about 5 wt % to about 90 wt %, about 10 wt % to about 90 wt %, about 25 wt % to about 80 wt %, about 30 wt % to about 70 wt %, or about 40 wt % to about 60 wt % HVLCM or sucrose acetate isobutyrate, based on weight of the vehicle or weight of the composition.
   13. The composition of any one of the preceding aspects, wherein the composition further comprises a solvent.
   14. The composition of any one of the preceding aspects, wherein the composition further comprises an organic solvent.
   15. The composition of any one of the preceding aspects, wherein the composition further comprises a hydrophilic solvent.
   16. The composition of any one of the preceding aspects, wherein the composition further comprises a hydrophobic solvent.
   17. The composition of aspect 13, wherein the solvent comprises at least one member selected from N-methyl-pyrrolidone (NMP), dimethylsulfoxide (DMSO), propylene carbonate (PC), benzyl alcohol (BA), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one.
   18. The composition of aspect 13, wherein the solvent comprises at least one member selected from N-methyl-pyrrolidone (NMP), dimethylsulfoxide (DMSO), propylene carbonate (PC), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one.
   19. The composition of aspect 13, wherein the solvent comprises at least one of N-methyl-pyrrolidone (NMP), dimethylsulfoxide (DMSO), propylene carbonate (PC), benzyl alcohol (BA), benzyl benzoate (BB), ethanol, and glycofurol.
   20. The composition of aspect 13, wherein the solvent comprises propylene carbonate (PC).
   21. The composition of any one of the preceding aspects, wherein the composition does not comprise N-methyl-pyrrolidone (NMP) and/or does not comprise ethanol.
   22. The composition of any one of the preceding aspects, wherein the composition comprises from about 10 wt % to about 90 wt %, about 10 wt % to about 80 wt %, about 10 wt % to about 60 wt %, about 10 wt % or about 40 wt %, or about 15 wt % to about 35 wt % solvent, based on weight of the vehicle or weight of the composition.
   23. The composition of any one of the preceding aspects, wherein the composition further comprises a polymer.
   24. The composition of aspect 23, wherein the polymer is linear or branched.
   25. The composition of aspects 23 or 24, wherein the polymer comprises a homopolymer.
   26. The composition of any one of aspects 23-25, wherein the polymer comprises a copolymer.
   27. The composition of any one of aspects 23-26, wherein the polymer comprises a lactic acid-based polymer.
   28. The composition of any one of aspects 23-27, wherein the polymer comprises an alkoxy end group, an acid end group, or hydroxyl end group.
   29. The composition of any one of aspects 23-28, wherein the polymer comprises an alkoxy end group that consists of 2 to 24 carbons or 8 to 24 carbons.
   30. The composition of aspect 29, wherein the alkoxy end group consists of 12 carbons.
   31. The composition of any one of aspects 23-30, wherein the polymer is initiated with a member selected from fatty alcohol and diol.
   32. The composition of any one of aspects 23-31, wherein the polymer is initiated with 1,6-hexanediol.
   33. The composition of any one of aspects 23-31, wherein the polymer is initiated with dodecanol.
   34. The composition of any one of aspects 23-33, wherein the polymer comprises poly(lactic acid)(glycolic acid).
   35. The composition of any one of aspects 23-34, wherein the polymer comprises lactic acid repeat units and glycolic acid repeat units in a molar ratio of from about 50:50 to about 100:0, about 70:30 to about 100:0, about 75:25 to about 100:0, or about 85:15 to about 95:5.
   36. The composition of any one of aspects 23-35, wherein the polymer has a weight average molecular weight of less than about 50,000 Daltons, less than about 40,000 Daltons, or less than about 30,000 Daltons, or from about 1000 Daltons to about 50,000 Daltons, about 4000 Daltons to about 40,000 Daltons, about 6000 Daltons to about 30,000 Daltons, about 10,000 Daltons to about 25,000 Daltons, or about 15,000 Daltons to about 20,000 Daltons.
   37. The composition of any one of aspects 23-35, wherein the polymer has a weight average molecular weight of less than about 70,000 Daltons, less than about 60,000 Daltons, less than about 50,000 Daltons, less than about 40,000 Daltons, or less than about 30,000 Daltons, or from about 1000 Daltons to about 50,000 Daltons, about 4000 Daltons to about 40,000 Daltons, about 6000 Daltons to about 30,000 Daltons, about 10,000 Daltons to about 25,000 Daltons, or about 15,000 Daltons to about 20,000 Daltons.
   38. The composition of any one of aspects 23-37, wherein the polymer has a weight average molecular weight after gamma irradiation that is from about 85% to about 99.9%, from about 90% to about 99%, or from about 95% to about 98%, relative to the weight average molecular of the polymer before gamma irradiation.
   39. The composition of any one of aspects 23-38, wherein the composition comprises less than about 40 wt %, less than about 30 wt %, less than about 20 wt %, or less than about 10 wt %, or from about 1 wt % to about 40 wt %, about 2 wt % to about 30 wt %, about 3 wt % to about 20 wt %, or about 5 wt % to about 10 wt % polymer, based on weight of the vehicle or weight of the composition.
   40. The composition of any one of the preceding aspects, wherein the composition does not comprise cellulose acetate butyrate.
   41. The composition of any one of aspects 23-40, wherein the weight ratio of the sucrose acetate isobutyrate to the polymer to the solvent is about 1: 0.1-2: 0.3-10, or 1: 0.2-1: 0.4-5, or 1: 0.3-0.5: 0.5-1.
   42. The composition of any one of the preceding aspects, wherein the vehicle is monophasic when stored at 25° C. for 7 days.
   43. The composition of any one of the preceding aspects, wherein the vehicle is monophasic when stored at 25° C. for 1 month.
   44. The composition of any one of the preceding aspects, wherein the composition has a viscosity of less than 10,000 cP at a shear rate of 100 s⁻¹ at 25° C.
   45. The composition of any one of the preceding aspects, wherein the composition has a viscosity of less than 20,000 cP or less than 10,000 cP at a shear rate of 100 s⁻¹ at 25° C.
   46. The composition of any one of the preceding aspects, wherein the composition has a viscosity of from about 50 cP to about 8000 cP or about 500 cP to about 6000 cP at a shear rate of 150 s⁻¹ at 25° C.
   47. The composition of any one of the preceding aspects, wherein the composition further comprises at least one member selected from viscosity enhancers, antioxidants, preservatives, and particle stabilizers.
   48. The composition of any one of the preceding aspects, wherein the composition comprises from about 0.001 wt % to about 0.35 wt % water, based on total weight of the composition.
   49. The composition of any one of the preceding aspects, wherein when the composition is placed in phosphate buffered saline at 37° C. (e.g., at pH 6.0 or 7.4), the amount of active agent released from the composition after 4 weeks is from about 20% to about 100%, about 20% to about 80%, about 40% to about 100%, about 50% to about 100%, or about 40% to about 80% of the total amount of the active agent in the composition.
   50. The composition of any one of the preceding aspects, wherein when the composition is placed in phosphate buffered saline at 37° C. (e.g., at pH 6.0 or 7.4), the amount of active agent released from the composition after 24 hours is less than about 40%, less than about 30%, less than about 20%, or less than about 10% of the amount released after 28 days.
   51. The composition any one of the preceding aspects, wherein when the composition is placed in phosphate buffered saline at 37° C. (e.g., at pH 6.0 or 7.4), the amount of active agent released after 28 days is greater than about 30%, greater than about 40%, greater than about 50%, greater than about 60%, greater than about 70%, or greater than about 80% of a total amount of active agent in the composition.
   52. The composition any one of the preceding aspects, wherein when the composition is placed in phosphate buffered saline at 37° C. (e.g., at pH 6.0 or 7.4), the amount of active agent released after 28 days is greater than about 20%, greater than about 30%, greater than about 40%, greater than about 50%, greater than about 60%, greater than about 70%, or greater than about 80% of a total amount of active agent in the composition.
   53. The composition of any one of the preceding aspects, wherein the composition has been sterilized.
   54. The composition of any one of the preceding aspects, wherein the composition has been gamma-irradiated.
   55. The composition of aspect 54, wherein the composition has been exposed to an average gamma irradiation dose of less than about 25 kGy.
   56. The composition of aspect 54 or 55, wherein the composition has been exposed to an average gamma irradiation dose from about 15 kGy to about 25 kGy.
   57. The composition of any one of aspects 54-56, wherein, after gamma-irradiation, the composition comprises at least 95%, at least 97%, at least 98%, or at least 99% of the active agent, relative to the amount of the active agent before gamma irradiation.
   58. The composition of any one of aspects 54-57, wherein, after gamma-irradiation, the composition comprises no more than about 5%, no more than about 3%, no more than about 2%, or no more than about 1% additional degradation products, relative to the amount of degradation products before gamma irradiation.
   59. The composition of any one of the preceding aspects, wherein the composition does not comprise risperidone.
   60. The composition of any one of the preceding aspects, wherein the composition achieves a therapeutically effective plasma concentration of the active agent, or a metabolite thereof, for at least about 7 days, about 14 days, about 21 days, about 28 days, or more, when the composition is administered subcutaneously as a single dose to a subject.
   61. The composition of any one of the preceding aspects, wherein the composition has been stored.
   62. The composition of any one of the preceding aspects, wherein the composition has been stored at from about 0° C. to about 20° C., about 1° C. to about 10° C., or about 2° C. to about 8° C.
   63. A unit dosage form comprising the composition of any one of the preceding claims.
   64. The unit dosage form of aspect 63, wherein the composition is contained within a vial.
   65. The unit dosage form of aspect 63, wherein the composition is contained within a syringe.
   66. The unit dosage form of aspect 63, wherein the composition is contained within a needle-free injector.
   67. A receptacle containing the composition of any one of aspects 1-62.
   68. A needle-free injector comprising the composition of any one of aspects 1-62.
   69. A composition as defined in any one of aspects 1-62 for use as a medicament.
   70. A composition as defined in any one of aspects 1-62 for use in a method of treating and/or preventing HIV and/or HBV infection.
   71. Use of a composition as defined in any one of aspects 1-62 for the manufacture of a medicament for treating and/or preventing HIV and/or HBV infection.
   72. A process of sterilizing the composition of any one of aspects 1-62, comprising gamma-irradiating the composition.
   73. A method of administering a therapeutically effective dose of a active agent to a subject in need thereof, the method comprising administering to the subject the composition of any one of aspects 1-62 or the unit dosage form of any one of aspects 63-66.
   74. The method of aspect 73, wherein the administration achieves a plasma active concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer.
   75. The method of aspect 73 or 74, wherein the administration comprises injecting the composition into the subject.
   76. The method of any one of aspects 73-75, wherein the composition or unit dosage form has been established to achieve a plasma active concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer in a dog subject.
   77. The method of any one of aspects 73-76, wherein the composition is administered at a frequency ranging from once a week to once every three months.
   78. The method of any one of aspects 73-77, wherein the administering comprises administering the composition or unit dosage form subcutaneously.
   79. The method of any one of aspects 73-78, wherein the composition is self-administered.
   80. The method of any one of aspects 73-79, wherein the composition is administered by a non-health care professional.
   81. The method of any one of aspects 73-80, wherein the composition is administered with a needle and syringe.
   82. The method of aspect 81, wherein the needle has a length of less than or equal to 1 inch.
   83. The method of aspect 81, wherein the needle has a length of less than or equal to ⅝ inch.
   84. The method of aspect 81, wherein the needle has a length of less than or equal to 0.5 inch.
   85. The method of any one of aspects 73-84, wherein the composition is administered with a pre-filled syringe or an auto-injector.
   86. The method of any one of aspects 73-85, wherein the composition is administered once a month.
   87. The method of any one of aspects 73-86, wherein the subject is receiving or has received an additional therapeutic agent.
   88. A method of treating and/or preventing HIV infection, the method comprising administering the composition of any one of aspects 1-62 or the unit dosage form of any one of aspects 63-66.
   89. A method of treating and/or preventing HBV infection, the method comprising administering the composition of any one of aspects 1-62 or the unit dosage form of any one of aspects 63-66.
   90. A method of manufacturing the composition of any one of the preceding aspects, comprising:

(a) providing the active agent; and

(b) combining the active agent with the vehicle to form the composition.

91. The method of aspect 90, further comprising reducing the amount of water in the composition.
92. The method of aspect 91, wherein the reducing the amount of water in the composition comprises placing the mixture under an inert gas, such as nitrogen.
93. The method of any one of aspects 90-92, further comprising heating the mixture.
94. The method of any one of aspects 90-93, further comprising mixing the mixture.

### EXAMPLES

### Abbreviations

ACN acetonitrile
BB benzyl benzoate
DCM dichloromethane
DD dodecanol
DMSO dimethylsulfoxide
EtOH ethanol
ND not determined
NMP N-methyl-2-pyrrolidone
PC propylene carbonate
PLA poly(lactic acid)
PLA-0 PLA with C₆-C₁₂ aliphatic chain ester end group (MW: <20 kDa)
PLA-1 DL-PLA lactic acid terminated (MW: 14 kDa)
PLA-2 DL-PLA lactic acid terminated (MW: 16 kDa)
PLA-3 DL-PLA initiated with 1-dodecanol (MW: 16 kDa)
PLA-4 DL-PLA (MW: 13 kDa)
PLGA poly(lactic acid)(glycolic acid)
50-50 PLGA-DD 50:50 DL-PLGA initiated with 1-dodecanol (MW: 7 kDa)
50-50 PLGA-LA 50:50 DL-PLGA lactic acid terminated (MW: 6 kDa)
65-35 PLGA-DD 65:35 DL-PLGA initiated with 1-dodecanol (MW: 7 to 8 kDa)
65-35 PLGA-2 65:35 PLGA initiated with 1-dodecanol (MW: 48.4 kDa)
75-25 PLGA 75:25 DL-PLGA initiated with 1-dodecanol (MW: 8 kDa)
75-25 PLGA-2 75:25 PLGA initiated with 1-dodecanol (MW: 40 kDa)
75-25 PLGA-3 75:25 PLGA initiated with 1-dodecanol (MW: 51 kDa)
75-25 PLGA-4 75:25 PLGA initiated with 1-dodecanol (MW: 29 kDa)
85-15 PLGA 85:15 PLGA terminated with hydroxyacid (MW: 40-80 kDa)
90-10 PLGA-1 90:10 DL-PLGA initiated with 1-dodecanol (MW: 18 kDa)
90-10 PLGA-2 90:10 DL-PLGA initiated with 1-dodecanol (MW: 8 kDa)
90-10 PLGA-3 90:10 DL-PLGA initiated with 1-dodecanol (MW: 11 kDa)
SAIB sucrose acetate isobutyrate
TAF tenofovir alafenamide
TFV-DP tenofovir diphosphate

### Example 1. Preparation of Vehicle Compositions

A representative method of making a formulation comprising SAIB, lactic acid-based polymer, and solvent follows:

SAIB was heated in a 60° C. oven. Solvent was weighed into a container with a stir bar. Lactic acid-based polymer(s) were added to the solvent with stirring until dissolution was achieved. Heated SAIB was added and stirred until a homogeneous composition was obtained. In some cases, the resulting vehicle was filtered through a flat sheet filter with a pore size of 5 microns.

Another representative method of making a formulation comprising SAIB, lactic acid-based polymer, and solvent follows:

Poly(lactic acid)(glycolic acid) (PLGA) was removed from cold storage and allowed to warm to room temperature. The polymer was weighed in a glass jar. Next, propylene carbonate (PC) was dispensed into the glass jar. To dissolve the PLGA in the PC, the mixture was placed in a rotator and rotated at 20 rpm at room temperature for about 12 hours. SAIB was heated to 80° C. for approximately an hour. The heated SAIB was poured into the glass jar containing the PLGA and PC. The mixture was rotated in an oven at 50° C. at 20 rpm for about 2 hours. The jar was removed from the oven and allowed to cool to room temperature.

Vehicle compositions were prepared according to Table 1A and Table 1B below. The viscosities of the vehicles were measured at a shear rate of 100 s⁻¹ to 500 s⁻¹ at 25° C. Unless noted otherwise, the vehicles remained as a single phase when maintained at 25° C. for a one week period.

TABLE 1A

| | | Vehicle |
|---|---|---|
| Vehicle | | Viscosity |
| No. | Vehicle (w/w/w %) | (25° C., cP) |
| V1 | SAIB/NMP/85-15 PLGA (65/25/10) | |
| V2 | SAIB/EtOH/PLA-0 (65/25/10) | |
| V3 | SAIB/NMP/75-25 PLGA (50/30/20) | 411 |
| V4 | SAIB/DMSO/75-25 PLGA (48/32/20) | 435 |
| V5 | SAIB/PC/75-25 PLGA (44/37/19) | 384 |
| V6 | SAIB/BB/75-25 PLGA (30/55/15) | 255 |
| V7 | SAIB/BB/PLA-1 (15/70/15) | 186 |
| V8 | SAIB/PC/PLA-3 (33/47/20) | 260 |
| V9 | SAIB/PC/65-35 PLGA-DD (44/37/19) | 332 |
| V10 | SAIB/NMP/BB/90-10 PLGA-1 (35/25/25/15) | 218 |
| V11 | SAIB/PC/90-10 PLGA-1 (40/45/15) | 252 |
| V12 | SAIB/BB/65-35 PLGA-DD (45/50/5) | 115 |
| V13 | SAIB/PC/BB/90-10 PLGA-1 (30/27.5/27.5/15) | 226 |
| V14 | SAIB/PC/BB/90-10 PLGA-1 (40/25/25/10) | 170 |
| V15 | SAIB/BB/90-10 PLGA-1 (10/70/20) | 446 |
| V16 | SAIB/PC/75-25 PLGA (44/37/19) | 384 |
| V17 | SAIB/BB/PLA-1 (15/70/15) | 186 |
| V18 | SAIB/BB/PLA-1 (15/70/15) | 186 |
| V19 | SAIB/BB/90-10 PLGA-1 (10/70/20) | 446 |
| V22 | SAIB/NMP/75-25 PLGA (50/30/20) | 411 |
| V23 | SAIB/DMSO/75-25 PLGA (48/32/20) | 435 |
| V24 | SAIB/NMP/75-25 PLGA (50/30/20) | 411 |
| V25 | SAIB/DMSO/75-25 PLGA (48/32/20) | 435 |
| V26 | SAIB/PC/75-25 PLGA (44/37/19) | 384 |
| V27 | SAIB/PC/75-25 PLGA (53/28/19) | 1435 |
| V28 | SAIB/PC/75-25 PLGA (33/37/30) | 1004 |
| V29 | SAIB/PC/90-10 PLGA (44/37/19) | 995 |
| V30 | SAIB/PC/75-25 PLGA (44/37/19) | 384 |
| V31 | SAIB/DMSO/75-25 PLGA (55/25/20) | 1212 |
| V32 | SAIB/DMSO/75-25 PLGA (38/32/30) | 962 |
| V33 | SAIB/DMSO/PLA-3 (48/32/20) | 789 |
| V34 | SAIB/DMSO/75-25 PLGA (48/32/20) | 435 |
| V35 | SAIB/BB/PLA-2 (43/42/15) | 1664 |
| V36 | SAIB/NMP/75-25 PLGA (57/23/20) | 1365 |
| V37 | SAIB/BB/PLA-3 (43/42/15) | 1331 |
| V38 | SAIB/BB/90-10 PLGA-1 (30/50/20) | 1931 |
| V39 | SAIB/BB/DMSO/90-10 PLGA-1 (34/40/6/20) | 1452 |
| V40 | SAIB/NMP/90-10 PLGA-1 (50:30:20) | 1032 |
| V41 | SAIB/PC/PLA-2 (44/37/19) | 907 |
| V42 | SAIB/PC/65-35 PLGA-DD (53/28/19) | 1221 |
| V43 | SAIB/PC/75-25 PLGA (62/28/10) | 639 |
| V44 | SAIB/PC/75-25 PLGA (53/32/15) | 583 |
| V45 | SAIB/PC/BB/75-25 PLGA (43.5/37/0.5/19) | 403 |
| V46 | SAIB/PC/BB/75-25 PLGA (43.5/36/1.5/19) | 388 |
| V47 | SAIB/PC/BB/75-25 PLGA (43/35/3/19) | 422 |
| V48 | SAIB/PC/IPM/75-25 PLGA (43/36/2/19) | 374 |
| V49 | SAIB/PC/50-50 PLGA-DD/PLA-2 (44/37/5/14) | 787 |
| V50 | SAIB/PC/50-50 PLGA-DD (53/28/19) | |
| V51 | SAIB/PC/50-50 PLGA-DD (44/37/19) | 427 |
| V52 | SAIB/PC/90-10 PLGA-2 (53/28/19) | 1176 |
| V53 | SAIB/PC/50-50 PLGA-LA (43/37/19) | |
| V54 | SAIB/PC/PLA-2/50-50 PLGA-LA (44/37/17.5/1.5) | 911 |
| V55 | SAIB/PC/PLA/50-50 PLGA-LA (44/37/14/5) | 777 |
| V56 | SAIB/PC/75-25 PLGA/50-50 PLGA-LA (44/37/17.5/1.5) | 441 |
| V57 | SAIB/PC/75-25 PLGA/50-50 PLGA-LA (44/37/14/5) | 471 |
| V58 | SAIB/PC/90-10 PLGA-1 (62/28/10) | 1092 |
| V59 | SAIB/PC/PLA-3 (44/37/19) | 871 |
| V60 | SAIB/PC/75-25 PLGA (44/37/19) | 384 |
| V61 | SAIB/PC/90-10 PLGA-1 (44/37/19) | 995 |
| V62 | SAIB/PC/90-10 PLGA-1 (44/37/19) | 995 |
| V63 | SAIB/PC/PLA-3 (32/49/19) | 272 |
| V64 | SAIB/PC/BB/PLA-3 (31.5/48/1.5/19) | 265 |
| V65 | SAIB/PC/90-10 PLGA-1 (62/28/10) | 1092 |
| V66 | SAIB/PC/PLA-3 (41/49/10) | 101 |
| V67 | SAIB/PC/PLA-3 (23/58/19) | 61 |
| V68 | SAIB/PC/PLA-3 (23/58/19) | 61 |
| V69 | SAIB/PC/90-10 PLGA-1 (52/33/15) | 974 |
| V70 | SAIB/PC/90-10 PLGA-1 (71/24/5) | 958 |
| V71 | SAIB/PC/90-10 PLGA-1 (60/30/10) | 753 |
| V72 | SAIB/PC/90-10 PLGA-1 (44/37/19) | 995 |
| V73 | SAIB/PC (79/21) | 959 |
| V74 | SAIB/PC/NMP/90-10 PLGA-1 (46/27/8/19) | 928 |
| V75 | SAIB/PC/NMP/90-10 PLGA-1 (61.5/22/6.5/10) | 734 |
| V76 | SAIB/PC/DMSO/90-10 PLGA-1 (61/22/7/10) | 727 |
| V77 | SAIB/PC/90-10 PLGA-1 (44/37/19) | 995 |
| V78 | SAIB/PC/90-10 PLGA-1 (60/30/10) | 753 |
| V79 | SAIB/PC/PLA-3 (44/37/19) | 728 |
| V80 | SAIB/PC/PLA-3 (60/30/10) | 656 |
| V81 | SAIB/PC/PLA-3 (44/37/19) | 728 |
| V82 | SAIB/PC/PLA-3 (44/37/19) | 728 |
| V83 | SAIB/PC/90-10 PLGA-1/PLA-3 (60/30/5/5) | 707 |
| V84 | SAIB/PC/90-10 PLGA-1 (52/33/15) | 974 |
| V85 | SAIB/PC/90-10 PLGA-1 (71/24/5) | 958 |
| V90 | SAIB/NMP/75-25 PLGA (50/30/20) | 403 |
| V91 | SAIB/PC/75-25 PLGA (44/37/19) | 412 |
| V92 | SAIB/BB/75-25 PLGA (40/40/20) | 1623 |
| V93 | SAIB/DMSO/75-25 PLGA (48/32/20) | 429 |
| V94 | SAIB/BB/PLA-4 (20/60/20) | 639 |
| V95 | SAIB/NMP/75-25 PLGA (20/60/20) | 34 |
| V96 | SAIB/PC/PLGA (20/60/20) (75:25 PLGA) | 73 |
| V97 | SAIB/BB/75-25 PLGA (20/60/20) | 318 |
| V98 | SAIB/DMSO/PLGA (20/60/20) (75:25 PLGA) | 40 |
| V99 | SAIB/BA/EtOH/75-25 PLGA (20/30/20/20) | 15 |
| V100 | SAIB/NMP/65-35 PLGA (50/30/20) | 409 |
| V101 | SAIB/NMP/90-10 PLGA-3 (50/30/20) | 585 |
| V102 | SAIB/GF/75-25 PLGA (44/37/19) | 1141 |
| V103 | SAIB/GF/75-25 PLGA (20/60/20) | 270 |
| V104 | SAIB/NMP/90-10 PLGA-2 (50/30/20) | |
| V105 | SAIB/PC/90-10 PLGA-2 (44/37/19) | |
| V106 | SAIB/NMP/75-25 PLGA (50/30/20) | |
| V107 | SAIB/PC/75-25 PLGA (44/37/19) | |
| V108 | SAIB/DMSO/PLGA (48/32/20) | |
| V109 | SAIB/BB/PLA-4 (20/60/20) | |
| V110 | SAIB/DMSO/75-25 PLGA (20/60/20) | |
| V111 | SAIB/NMP/90-10 PLGA-3 (50/30/20) | |

TABLE 1B

| Vehicle | | Vehicle Viscosity |
|---|---|---|
| No. | Vehicle (w/w/w %) | (25° C., cP) |
| V112 | SAIB/PC/65-35 PLGA-2 (25/55/20) | 1867 |
| V113 | SAIB/PC/90-10 PLGA-1 (38/37/25) | 1882 |
| V114 | SAIB/PC/DMSO/90-10 PLGA-1 (50/21/9/20) | 1990 |
| V115 | SAIB/PC/EtOH/90-10 PLGA-1 (53/21.5/5.5/20) | 1530 |
| V116 | SAIB/PC/75-25 PLGA-2 (34/46/20) | 2108 |
| V117 | SAIB/PC/75-25 PLGA-3 (25/55/20) | 1951 |
| V118 | SAIB/PC/75-25 PLGA-4 (37/43/20) | 1780 |

### Example 2. Solubility of Tenofovir Agents

The release of an active agent from SAIB/polymer/solvent formulations (e.g., provided vehicle formulations) depends on a variety of factors, including the solubility of the active agent in the solvent of provided formulations. This Example demonstrates the solubility of TAF free base and in salt forms with various solvents.

Briefly, samples were prepared by mixing active agent and solvent and were tested at various time points after mixing and after storage at various temperatures. The samples were tested for the solubility of the active agent (mg/mL), calculated as the free base equivalent. The results and compositions tested are shown in Table 2. Each data point is the average of two replicates unless otherwise specified. The data points with an asterisk (*) were taken at 4 days as opposed to 5 days.

TABLE 2

| | Solubility (mg/mL, Calculated as Free Base Equivalent) | | | | |
|---|---|---|---|---|---|
| | | | TAF | TAF | TAF |
| | TAF Free base | | Hemifumarate | Hemipamoate | Sebacate |
| Sol- | 37° C./1 | 37° C./5 | 37° C./5 | 37° C./5 | 37° C./ |
| vent | day | days | days | days | 5 days |
| DCM | 146 | 384 | 97 | 220 | 216 |
| DMSO | 96 | 182 | 132 | 203 | 109 |
| NMP | 96 | 161 | 160 | 93 | 88 |
| ACN | 15 | 13 | 11 | 8 | 3 |
| PC | 15 | 26 | 22 | 46 | 6 |
| BB | Not | 5.0 (n = 1)* | 3.9* | 1.5* | 2.2* |
| | deter- | | | | |
| | mined | | | | |

The present disclosure encompasses the recognition that solvents can be chosen in order to achieve a desirable release rate from the formulation. In some embodiments, a solvent is chosen in which the active agent is less soluble in order to provide a slower release rate from the formulation. In some embodiments, a solvent is chosen in which the active agent is more soluble in order to provide a faster release rate from the formulation.

### Example 3. Preparation of TAF Compositions

Provided compositions were prepared according to the following general procedure: Tenofovir agent was added to a vehicle composition (prepared as described in Example 1), followed by homogenization. Before being combined with the vehicle, the active agent typically had a d90 particle size of about 20 to 30 microns, except for formulation F59 which used TAF sebacate milled to a d90 particle size of about 3 microns.

The formulations described in Table 3A and Table 3B were prepared according to the above procedure. In Table 3A and Table 3B, % active loading (w/w) is based on TAF free base equivalence values. The viscosities of the formulations were measured at a shear rate of 100 s⁻¹ to 500 s⁻¹ at 25° C.

TABLE 3A

| | | % Active | | | Form. |
|---|---|---|---|---|---|
| Form. | | Loading | Solution or | | Viscosity |
| No. | Active Agent | (w/w) | Suspension | Vehicle | (25° C., cP) |
| F1 | TAF sebacate | 8.6 | Suspension | V1 | |
| F2 | TAF free base | 8.6 | Solution | V2 | |
| F3 | TAF free base | 8.6 | Suspension | V1 | |
| F4 | TAF free base | 7.8 | Suspension | V3 | 618 |
| F5 | TAF free base | 7.8 | Solution | V4 | 725 |
| F6 | TAF free base | 7.8 | Suspension | V5 | 553 |
| F7 | TAF free base | 7.8 | Suspension | V6 | 371 |
| F8 | TAF free base | 7.8 | Suspension | V7 | 292 |
| F9 | TAF free base | 7.8 | Suspension | V8 | 390 |
| F10 | TAF free base | 7.8 | Suspension | V9 | 503 |
| F11 | TAF free base | 7.8 | Suspension | V10 | 316 |
| F12 | TAF free base | 7.8 | Suspension | V11 | 367 |
| F13 | TAF free base | 7.8 | Suspension | V12 | 196 |
| F14 | TAF free base | 7.8 | Suspension | V13 | 345 |
| F15 | TAF free base | 7.8 | Suspension | V14 | 262 |
| F16 | TAF free base | 7.8 | Suspension | V15 | 630 |
| F17 | TAF sebacate | 7.8 | Suspension | V16 | 793 |
| F18 | TAF sebacate | 7.8 | Suspension | V17 | 492 |
| F19 | TAF hemipamoate | 7.8 | Suspension | V18 | 301 |
| F20 | TAF hemipamoate | 7.8 | Suspension | V19 | 673 |
| F23 | TAF sebacate | 7.8 | Suspension | V22 | |
| F24 | TAF sebacate | 7.8 | Solution | V23 | 758 |
| F25 | TAF hemipamoate | 7.8 | Solution | V24 | |
| F26 | TAF hemipamoate | 7.8 | Solution | V25 | |
| F27 | TAF hemipamoate | 7.8 | Suspension | V26 | |
| F28 | TAF sebacate | 7.8 | Suspension | V27 | 2609 |
| F29 | TAF sebacate | 7.8 | Suspension | V28 | 2070 |
| F30 | TAF sebacate | 7.8 | Suspension | V29 | 2063 |
| F31 | TAF sebacate | 15.6 | Suspension | V30 | 2317 |
| F32 | TAF free base | 7.8 | Suspension | V31 | 2036 |
| F33 | TAF free base | 7.8 | Solution | V32 | 1516 |
| F34 | TAF free base | 7.8 | Solution | V33 | 1344 |
| F35 | TAF free base | 15.6 | Suspension | V34 | 1126 |
| F36 | TAF hemipamoate | 7.8 | Suspension | V35 | 2445 |
| F37 | TAF free base | 7.8 | Suspension | V36 | 2123 |
| F38 | TAF | 7.8 | Suspension | V37 | 1940 |
| | hemipamoate | | | | |
| F39 | TAF hemipamoate | 7.8 | Suspension | V38 | 2770 |
| F40 | TAF hemipamoate | 7.8 | Suspension | V39 | 3358 |
| F41 | TAF free base | 7.8 | Suspension | V40 | 1626 |
| F42 | TAF sebacate | 7.8 | Suspension | V41 | 1912 |
| F43 | TAF sebacate | 7.8 | Suspension | V42 | 2304 |
| F44 | TAF sebacate | 7.8 | Suspension | V43 | 1283 |
| F45 | TAF sebacate | 7.8 | Suspension | V44 | 1222 |
| F46 | TAF sebacate | 7.8 | Suspension | V45 | 833 |
| F47 | TAF sebacate | 7.8 | Suspension | V46 | 853 |
| F48 | TAF sebacate | 7.8 | Suspension | V47 | 895 |
| F49 | TAF sebacate | 7.8 | Suspension | V48 | 775 |
| F50 | TAF sebacate | 7.8 | Suspension | V49 | 1478 |
| F51 | TAF sebacate | 7.8 | Suspension | V51 | 840 |
| F52 | TAF sebacate | 7.8 | Suspension | V52 | 2250 |
| F53 | TAF sebacate | 7.8 | Suspension | V53 | 743 |
| F54 | TAF sebacate | 7.8 | Suspension | V54 | 1687 |
| F55 | TAF sebacate | 7.8 | Suspension | V55 | 1568 |
| F56 | TAF sebacate | 7.8 | Suspension | V56 | 879 |
| F57 | TAF sebacate | 7.8 | Suspension | V57 | 937 |
| F58 | TAF sebacate | 7.8 | Suspension | V58 | 2280 |
| F59^{b} | TAF sebacate | 7.8 | Suspension | V58 | |
| F60 | TAF sebacate | 7.8 | Suspension | V59 | 1610 |
| F61 | TAF sebacate | 23.4 | Suspension | V60 | |
| F62 | TAF sebacate | 15.6 | Suspension | V61 | 4694 |
| F63 | TAF sebacate | 23.4 | Suspension | V62 | |
| F64 | TAF sebacate | 23.4 | | V63 | 14900 |
| F65 | TAF sebacate | 23.4 | | V64 | 16300 |
| F66 | TAF sebacate | 15.6 | Suspension | V65 | 5137 |
| F67 | TAF sebacate | 15.6 | | V66 | 13800 |
| F68 | TAF sebacate | 23.4 | | V67 | 14300 |
| F69 | TAF hemipamoate | 23.4 | NA | V68 | |
| F70 | TAF sebacate | 15.6 | Suspension | V69 | 3784 |
| F71 | TAF sebacate | 15.6 | Suspension | V70 | 4113 |
| F72 | TAF sebacate | 19.5 | Suspension | V71 | 5679 |
| F73 | TAF sebacate | 19.5 | Suspension | V72 | 6739 |
| F74 | TAF sebacate | 19.5 | Suspension | V73 | 7672 |
| F75 | TAF sebacate | 19.5 | Suspension | V74 | 4204 |
| F76 | TAF sebacate | 19.5 | Suspension | V75 | 3758 |
| F77 | TAF sebacate | 19.5 | Suspension | V76 | 3251 |
| F78 | TAF sebacate | 23.4 | Suspension | V77 | 7185 |
| F79 | TAF sebacate | 23.4 | Suspension | V78 | 5778 |
| F80 | TAF sebacate | 23.4 | Suspension | V79 | 2644 |
| F81 | TAF sebacate | 23.4 | Suspension | V80 | 4745 |
| F82 | TAF sebacate | 27.3 | Suspension | V81 | 8544 |
| F83 | TAF sebacate | 31.2 | | V82 | |
| F84 | TAF sebacate | 23.4 | Suspension | V83 | 5251 |
| F85 | TAF sebacate | 23.4 | Suspension | V84 | 6427 |
| F86 | TAF sebacate | 23.4 | Suspension | V85 | 6596 |
| F87 | TAF sebacate | 11.7 | Suspension | V86 | 1930 |

^{a}Phase separation observed.
^{b}Active agent was milled to a d90 particle size of about 3 microns prior to combining with vehicle.

TABLE 3B

| | | % Active | | | Form. |
|---|---|---|---|---|---|
| Form. | | Loading | Solution or | | Viscosity |
| No. | Active Agent | (w/w) | Suspension | Vehicle | (25° C., cP) |
| F88 | TAF sebacate | 7.8 | Suspension | V112 | |
| F89 | TAF sebacate | 7.8 | Suspension | V113 | |
| F90 | TAF sebacate | 7.8 | Suspension | V114 | |
| F91 | TAF sebacate | 7.8 | Suspension | V115 | |
| F92 | TAF sebacate | 7.8 | Suspension | V116 | 3009 |
| F93 | TAF sebacate | 7.8 | Suspension | V117 | 2639 |
| F94 | TAF sebacate | 7.8 | Suspension | V118 | 2178 |

### Example 4. Water Content of Vehicles and TAF Compositions

A vehicle composition including 62/28/10 (wt %) SAIB/PC/PLGA-2 was prepared as described in Example 1 above, and a formulation was prepared by using that vehicle in a formulation including 7.8 wt % TAF sebacate (calculated as the free base equivalent).

The vehicle was initially treated under 2 different conditions: (1) 5 g of the vehicle was transferred into 20 mL scintillation vials and the lids were removed. Half of the vials were left on a laboratory bench at ambient temperature, and half were stored in a reference standard chamber containing dessicator. (2) 20 g of the vehicle was transferred into a 60 mL jar. The vehicle was stirred in a 40° C. oven using stir bar and magnetic stirrers. Samples were taken at various timepoints and tested for water content before and after each treatment (Table 4).

TABLE 4

| | % Water | | | | | |
|---|---|---|---|---|---|---|
| | | | | Average | Std | % |
| Sample | n = 1 | n = 1 | n = 1 | (n = 3) | dev | RSD |
| T = 0 | 0.21 | 0.21 | 0.21 | 0.21 | 0.00 | 0.60 |
| Ambient, 48 hrs | 0.24 | 0.25 | 0.24 | 0.24 | 0.00 | 0.69 |
| Ambient with | 0.31 | 0.31 | 0.31 | 0.31 | 0.00 | 0.38 |
| Desiccators, 48 | | | | | | |
| hrs | | | | | | |
| Ambient, 96 hrs | 0.32 | 0.32 | 0.33 | 0.32 | 0.01 | 1.66 |
| Ambient with | 0.39 | 0.38 | 0.38 | 0.38 | 0.00 | 1.00 |
| Desiccators, 96 | | | | | | |
| hrs | | | | | | |
| 40° C., 8 hrs | 0.10 | 0.10 | 0.11 | 0.10 | 0.00 | 2.95 |
| 40° C. | 0.13 | 0.13 | 0.14 | 0.13 | 0.00 | 2.96 |
| Overnight | | | | | | |

Containers of the vehicle alone and formulation comprising active agent were placed into a glove box containing nitrogen and stirred overnight using stirs bars and magnetic stirrers. The vehicle and formulation comprising active agent were tested for water content both before and after exposure to nitrogen in the glove box, and samples were taken at various timepoints during the nitrogen exposure. Tables 5 and 6 below show the results for the vehicle and formulation, respectively.

TABLE 5

| | % Water | | | | |
|---|---|---|---|---|---|
| Sample | | | Average | | |
| (vehicle) | n = 1 | n = 1 | (n = 2) | Std dev | % RSD |
| T = 0 | 0.21 | 0.23 | 0.22 | 0.02 | 8.08 |
| T = 2 hrs | 0.20 | 0.19 | 0.19 | 0.00 | 1.97 |
| T = 6 hrs | 0.14 | 0.13 | 0.13 | 0.01 | 8.06 |
| T = 19 hrs | 0.06 | 0.04 | 0.05 | 0.01 | 16.20 |
| T = 25 hrs | 0.04 | 0.02 | 0.03 | 0.01 | 30.08 |

TABLE 6

| | % Water | | | | |
|---|---|---|---|---|---|
| Sample | | | Average | | |
| (formulation) | n = l | n = l | (n = 2) | Std dev | % RSD |
| T = 0 | 0.23 | 0.23 | 0.23 | 0.00 | 0.75 |
| T = 2 hrs | 0.19 | 0.20 | 0.19 | 0.00 | 1.02 |
| T = 6 hrs | 0.12 | 0.13 | 0.13 | 0.00 | 1.68 |
| T = 19 hrs | 0.04 | 0.05 | 0.04 | 0.00 | 8.13 |
| T = 25 hrs | 0.02 | 0.03 | 0.03 | 0.00 | 12.78 |

As can be seen from Tables 5 and 6, exposure to nitrogen surprisingly reduced the water content from 0.22% to 0.03% (vehicle alone), and from 0.23% to 0.03% (formulation).

### Example 5. Irradiation Effect on TAF Formulations

Injectable formulations are often irradiated to render them aseptic for use. This Example demonstrates the stability of the formulations following irradiation.

Representative samples were prepared as generally described in Example 3 above with components as summarized in Table 7. Samples were gamma irradiated at 15-20 kGy, and the samples were tested at various time points after irradiation and after storage at various temperatures. The samples were tested for the concentration of active agent in the sample. The concentration of active agent in the formulation was essentially unchanged by irradiation after storage under various conditions.

TABLE 7

| | | 2 wk, | 4 wk, | 4 wk, | Post |
|---|---|---|---|---|---|
| | t = 0 | 37° C. | 37° C. | 25° C. | irradiation |
| | Conc | Conc | Conc | Conc | Conc |
| Form. | (mg/g) | (mg/g) | (mg/g) | (mg/g) | (mg/g) |
| No. | Avg | Avg | Avg | Avg | Avg |
| F4 | 80.7 | 74.2 | 68.4 | 77.1 | 82.8 |
| F5 | 80.0 | 74.6 | 67.3 | 76.3 | 77.7 |
| F6 | 80.8 | 77.3 | 75.6 | 79.3 | 79.1 |
| F7 | 80.5 | 77.2 | 76.1 | 73.9 | 78.4 |
| F8 | 81.4 | 75.8 | 73.3 | 77.8 | 78.0 |
| F9 | 80.6 | 76.6 | 74.3 | 78.5 | 77.7 |
| F10 | 80.5 | 76.4 | 73.2 | 78.8 | 78.0 |
| F11 | 80.4 | 73.8 | 66.7 | | 78.5 |
| F12 | 80.4 | 77.7 | 75.9 | | 78.8 |
| F13 | 81.8 | 77.5 | 77.3 | 78.6 | 78.5 |
| F14 | 79.5 | 77.9 | 76.3 | | 78.4 |
| F15 | 79.9 | 77.5 | 75.8 | | 78.2 |
| F16 | 80.3 | 77.8 | 76.9 | | 78.4 |
| F17 | 79.8 | 77.5 | 83.7 | | 79.6 |
| F18 | 82.6 | 81.9 | 80.9 | | 80.3 |
| F19 | 81.7 | 81.0 | 79.6 | | 82.4 |
| F20 | 82.2 | 80.4 | 82.6 | | 81.9 |
| F24 | | | | | 79.9 |

To determine the effect of irradiation on the polymers in the formulation, the weight average molecular weight of the polymer was evaluated after various times and storage conditions following irradiation. Tables 8 and 9 show results of representative samples. As can be seen from Tables 8 and 9, irradiation does not significantly affect the degradation rate of polymers in tested formulations, but storage at increased temperatures resulted in degradation of polymers in some cases.

TABLE 8

| | MW (Da) | | |
|---|---|---|---|
| Form. | T = 0 | 1 = 0 | 2 wk 37° C. |
| No. | (nonirradiated) | (irradiated) | (nonirradiated) |
| F4 | 8302 | 7898 | 5808 |
| F5 | 8480 | 8201 | 5393 |
| F6 | 8893 | 8608 | 8189 |
| F7 | 8902 | 8821 | 8497 |
| F8 | 9093 | 9786 | 8877 |
| F9 | 15293 | 14409 | 13524 |
| F10 | 8058 | 7760 | 6962 |
| F11 | 16849 | 15597 | 8439 |
| F12 | 17952 | 16252 | 14606 |
| F13 | 8000 | 7959 | 7634 |
| F14 | 17914 | 17006 | 15242 |
| F15 | 18041 | 17086 | 14257 |
| F16 | 17666 | 17240 | 15920 |
| F17 | 8909 | 8660 | 8556 |
| F18 | 10863 | 10649 | 9989 |
| F19 | 11070 | 11067 | 10276 |
| F20 | 18054 | 17698 | 17408 |

TABLE 9

| | MW (% of T = 0) | | | |
|---|---|---|---|---|
| Form. | | 2 wk | 4 wk | 4 wk |
| No. | Irradiated | 37° C. | 37° C. | 25° C. |
| F4 | 95.1 | 70.0 | 53.8 | 79.5 |
| F5 | 96.7 | 63.6 | 43.6 | 69.4 |
| F6 | 96.8 | 92.1 | 82.2 | 96.1 |
| F7 | 99.1 | 95.5 | 88.7 | 97.5 |
| F8 | 107.6 | 97.6 | 88.9 | 100.4 |
| F9 | 94.2 | 88.4 | 76.0 | 93.7 |
| F10 | 96.3 | 86.4 | 72.4 | 91.9 |
| F11 | 92.6 | 50.1 | 35.9 | 66.3 |
| F12 | 90.5 | 81.4 | 67.0 | 90.8 |
| F13 | 99.5 | 95.4 | 89.9 | 97.0 |
| F14 | 94.9 | 85.1 | 71.8 | 91.7 |
| F15 | 94.7 | 79.0 | 64.2 | 88.6 |
| F16 | 97.6 | 90.1 | 78.3 | 93.5 |
| F17 | 97.2 | 96.0 | 90.4 | 100.3 |
| F18 | 98.0 | 92.0 | 81.2 | 97.0 |
| F19 | 100.0 | 92.8 | 82.6 | 98.7 |
| F20 | 98.0 | 96.4 | 90.8 | 99.2 |
| F24 | 94.4 | | | |

### Example 6. Injection Testing of TAF Compositions

The injection characteristics of certain formulations were investigated. Several formulations were tested for injection time using a 1 mL Exel syringe under 5 lbf, delivering 0.5 mL (nominally) volume and using 19 G 1″ (TW) needles. Two syringes were tested per formulation. The results are summarized in Table 10.

TABLE 10

| Form. | Injection |
|---|---|
| No. | Time (sec) |
| F62 | 5.0, 4.8 |
| F73 | 7.6, 7.7 |
| F78 | 11.5, 12.3 |
| F79 | 10.5, 11.0 |

The injection characteristics of formulation F58 were also investigated. Using an Instron materials tester and 3 mL BD disposable syringes with Terumo 19 G×⅝″ needles, two injection tests were performed on each of five vials of formulation for a total of five readings at each of two conditions: (1) to deliver 2.0 mL in 10 seconds using an injection speed of 3.5 mm/s; and (2) to deliver 0.2 mL in 5 seconds using an injection speed of 0.7 mm/s. After each injection test, the amount delivered was measured and recorded. The force during injection was reported from 40% to 80% of the distance traveled at the speed specified in the protocol.

Table 11 presents the results of injection test (1) with F58. The average glide force for the 2.0 mL injections was 43.70 Newtons. Table 12 presents the results of injection test (2) with F58. The average glide force for the 0.2 mL injections was 11.98 Newtons.

TABLE 11

| | Room | Glide | Volume | Time |
|---|---|---|---|---|
| | Temp. | Force | Delivered | Required |
| Run | (° C.) | (N) | (mL) | (s) |
| 1 | 24.2 | 45.29 | 2.07 | 10.3 |
| 2 | 24.4 | 42.95 | 2.05 | 10.3 |
| 3 | 24.3 | 41.72 | 2.05 | 10.3 |
| 4 | 23.6 | 43.73 | 2.03 | 10.2 |
| 5 | 23.9 | 44.80 | 2.01 | 10.1 |
| Average | 24.1 | 43.70 | 2.04 | 10.2 |

TABLE 12

| | Room | Glide | Volume | Time |
|---|---|---|---|---|
| | Temp. | Force | Delivered | Required |
| Run | (° C.) | (N) | (mL) | (s) |
| 1 | 24.0 | 11.90 | 0.21 | 5.3 |
| 2 | 24.1 | 11.01 | 0.21 | 5.2 |
| 3 | 24.3 | 12.79 | 0.20 | 5.1 |
| 4 | 24.0 | 11.33 | 0.21 | 5.4 |
| 5 | 24.1 | 12.86 | 0.21 | 5.2 |
| Average | 24.1 | 11.98 | 0.21 | 5.2 |

The force required at higher speed injection was surprisingly lower than the force required at lower speed injection. Specifically, the higher speed injection was five times faster than the lower speed injection (3.5 mm/s v. 0.7 mm/s). The force required at the higher speed injection (average of 43.70 Newtons) was less than five times the force required at the lower speed injection (average 11.98 Newtons).

### Example 7. In Vitro Release of TAF Compositions

The present disclosure provides formulations that allow for prolonged release when administered (e.g., via injection). This Example provides representative embodiments of formulations that achieve prolonged release.

Representative samples were prepared as generally described in Example 3 above with components as summarized in Table 13. The release of active agent from the samples was measured in aqueous buffer (Dulbecco's PBS, pH 7.4 or 20 mM KH₂PO₄, pH 6.0, 0.9% NaCl) at 37° C. Briefly, 0.5 mL of sample formulation (room temperature) was placed in a fixed surface area cup, which was moved to 100 mL fresh 37° C. buffer at each time point; gentle agitation was performed during release rate testing.

TABLE 13

| Form. | TAF Delivered 0-24 hours (%) | |
|---|---|---|
| No. | Average | St dev (n = 3) |
| F17 | 4.8 | 0.1 |
| F5 | 11.1 | 6.4 |
| F20 | 18.9 | 0.7 |
| F4 | 19.0 | 11.5 |
| F24 | 19.5 | 11.3 |
| F10 | 25.4 | 7.2 |
| F19 | 26.6 | 13.3 |
| F26 | 28.4 | 5.9 |
| F23 | 35.5 | 1.3 |
| F18 | 36.6 | 3.7 |
| F6 | 37.4 | 3.4 |
| F25 | 40.8 | 13.2 |
| F27 | 41.4 | 0.9 |
| F12 | 42.8 | 5.8 |
| F9 | 48.9 | 5.2 |
| F7 | 49.6 | 4.8 |
| F13 | 51.7 | 3.9 |
| F8 | 69.3 | 9.0 |
| F14 | 73.9 | 2.4 |
| F15 | 75.0 | 3.9 |
| F16 | 81.1 | 15.4 |
| F11 | 85.8 | 2.5 |

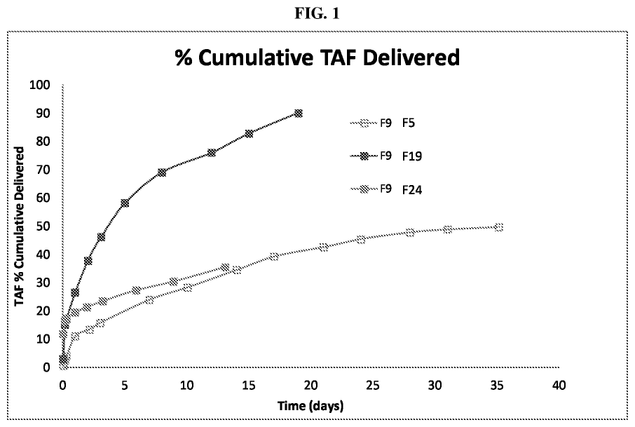

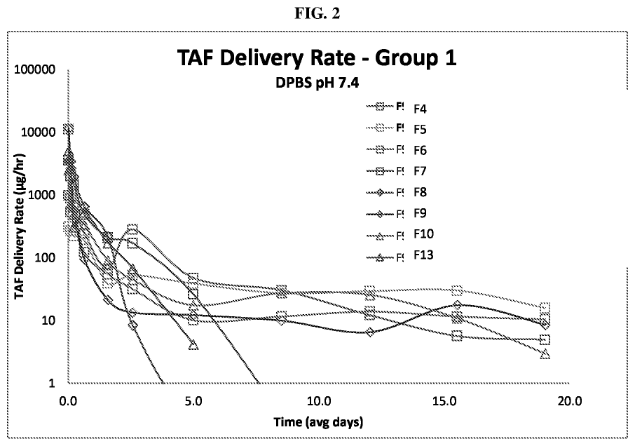

The cumulative release (%) of TAF from selected formulations of Table 13 is plotted in FIG. 1. For formulation F5 in FIG. 1, Dulbecco's PBS pH 7.4 buffer was used for the first 10 days. For all other time points in FIG. 1, 20 mM KH₂PO₄, pH 6.0, with 0.9% NaCl buffer was used. FIG. 2 depicts the delivery rate (μg/h) of TAF of selected formulations of Table 13. For formulations F4, F5, F6, F7 and F10 in FIG. 2, Dulbecco's PBS pH 7.4 buffer was used for the first 10 days. For all other time points in FIG. 2, 20 mM KH₂PO₄, pH 6.0, with 0.9% NaCl buffer was used.

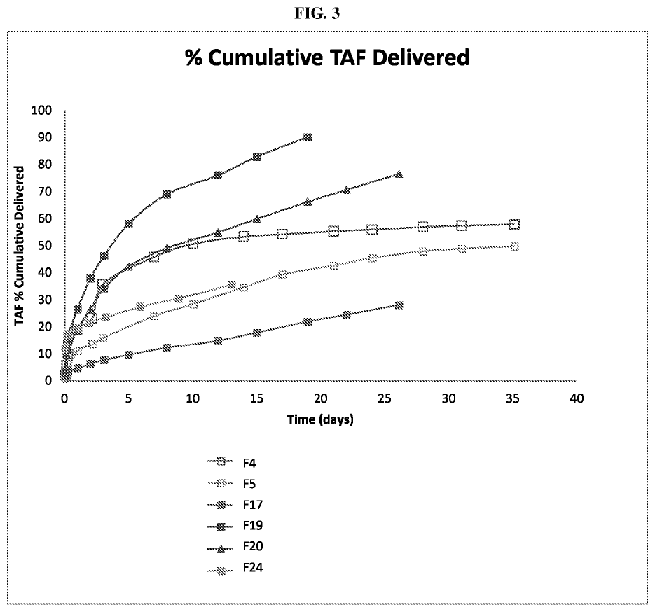

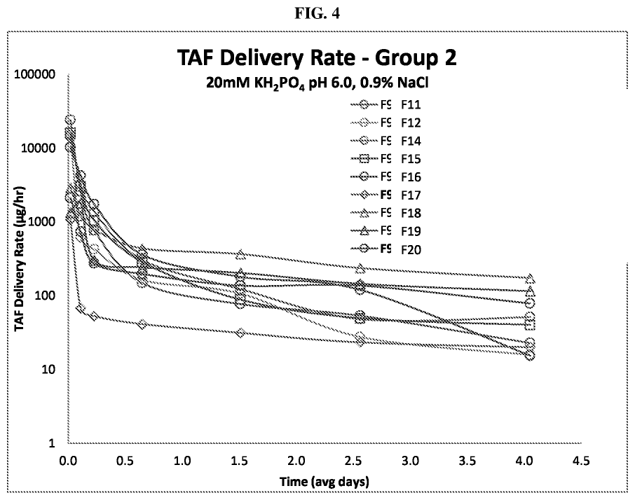

The cumulative release (%) of TAF from selected formulations of Table 13 is plotted in FIG. 3. For formulations F4 and F5 in FIG. 3, Dulbecco's PBS pH 7.4 buffer was used for the first 10 days. For all other time points in FIG. 3, 20 mM KH₂PO₄, pH 6.0, with 0.9% NaCl buffer was used. FIG. 4 depicts the delivery rate (μg/h) of TAF of selected formulations of Table 13 in 20 mM KH₂PO₄, pH 6.0, with 0.9% NaCl buffer.

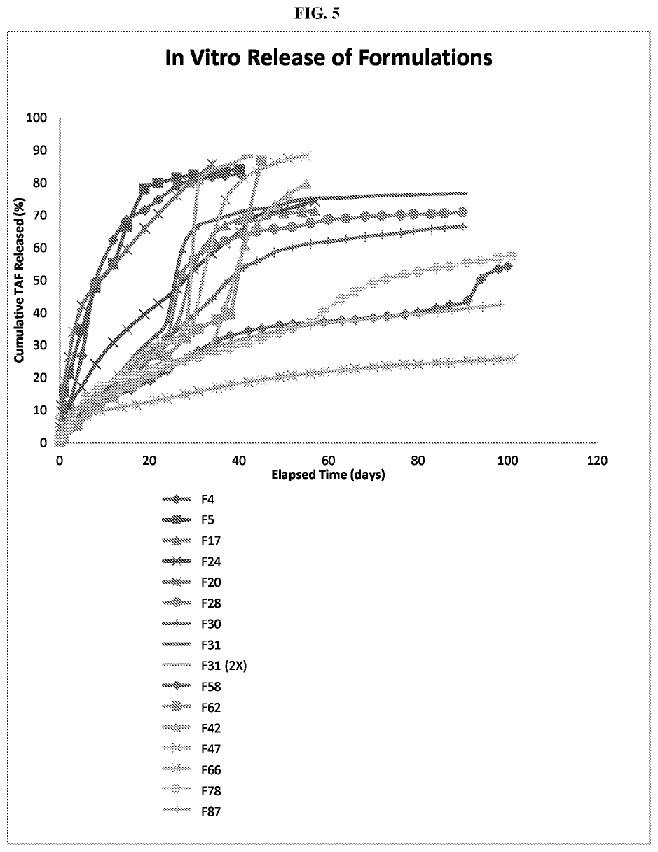

The cumulative release (%) of TAF from additional selected formulations of Table 13 is depicted in FIG. 5. For formulations F4 and F5 in FIG. 5, Dulbecco's PBS pH 7.4 buffer was used for the first 10 days. For all other time points in FIG. 5, 20 mM KH₂PO₄, pH 6.0, with 0.9% NaCl buffer was used.

### Example 8. In Vitro Release of Provided Compositions

The present disclosure provides formulations that allow for prolonged release when administered (e.g., via injection) and/or those that allow for desirable initial release profiles (e.g., to avoid an initial burst release of active agent). This Example provides representative embodiments of formulations that achieve prolonged release and/or desirable initial release profiles.

Representative samples were prepared as generally described in Example 3 above. The release of active agent from the samples was measured in aqueous buffer (Dulbecco's PBS, pH 7.4) at 37° C. Briefly, 0.5 mL of sample formulation (room temperature) was placed in a fixed surface area cup, which was moved to 100 mL fresh 37° C. buffer at each time point; gentle agitation was performed during release rate testing.

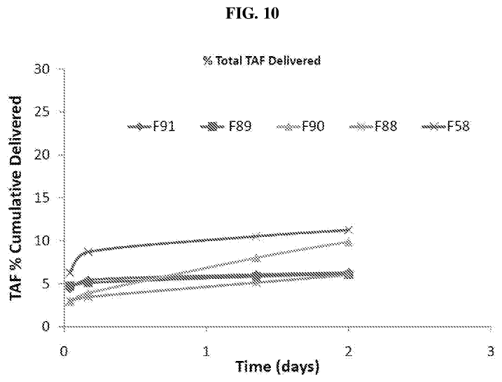

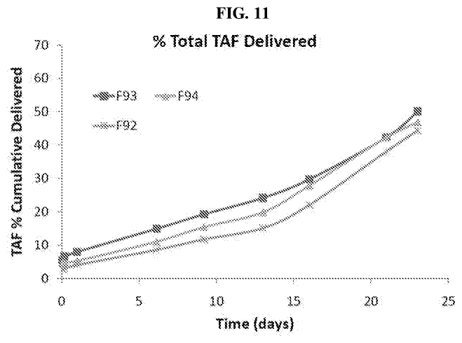

FIG. 10 shows cumulative release (%) of TAF from selected formulations of Table 3B over a 2-day period. FIG. 11 shows cumulative release (%) of TAF from selected formulations of Table 3B over a 24-day period.

### Example 9. Pharmacokinetics of TAF Compositions in Dogs

Provided compositions were administered via single subcutaneous (SC) injection to male beagle dogs (e.g., 0.5 mL provided formulation containing a dose of 45 mg TAF free base equivalent). Plasma and peripheral blood mononuclear cell (PBMC) samples were collected at certain time points postdose. Plasma concentrations of TAF and PBMC concentrations of TFV-DP were determined via LC-MS/MS, and the number of days was recorded for which a detectable level of TAF or TFV-DP was observed.

Table 14 summarizes the results of the PK evaluation in dogs.

TABLE 14

| | Detectable | TAF | Detectable | TFV-DP |
|---|---|---|---|---|
| | TAF | plasma | TFV-DP | intracellular |
| Form. | plasma level | LLOQ | intracellular | LLOQ |
| No. | (days)^{a} | (ng/mL)^{b} | level (days) | (nM)^{d} |
| F1 | 1.5 | 0.5 | ND | 10 |
| F2 | 3.3 | 0.5 | 6 | 10 |
| F3 | 2.2 | 0.5 | ND | 10 |
| F4 | 4.70 | 0.1 | ND | 10 |
| F5 | 4.0 | 0.1 | ND | 10 |
| F17 | 77.7 | 0.01 | 85.0 | 10 |
| F20 | 3.30 | 0.1 | 37.0 | 10 |
| F24 | 35.0 | 0.01 | ND | 10 |
| F28 | 34.0 | 0.01 | ND | 10 |
| F30 | 49.3 | 0.01 | 49.7 | 10 |
| F31 | 41.3 | 0.01 | 80.0 | 10 |
| F42 | 20.6 | 0.01 | ND | 10 |
| F47 | 26.0 | 0.01 | ND | 10 |
| F58 | 33.0 | 0.01 | 54.3 | 10 |
| F59 | 29.0 | 0.01 | 49.0 | 10 |
| F62 | 39.3 | 0.01 | 54.3 | 10 |
| F66 | 41.3 | 0.01 | 27 | 10 |
| F78 | 45.0 | 0.01 | 27 | 10 |
| F87 | 28.3 | 0.01 | ND | 10 |

^{a}Time of last detected TAF plasma concentration; all values are average of 3 dogs.
^{b}Lower limit of quantification for TAF plasma concentration determination by LC-MS/MS assay.
^{c}Time of last detected intracellular TFV-DP concentration in PBMCs; all values are average of 3 dogs.
^{d}Lower limit of quantification for intracellular TFV-DP concentration in PBMCs determined by LC-MS/MS assay.
LLOQ of cell counting assay was 2.0 million cells/sample.

### Example 10. Effect of Polymer Molecular Weight on Pharmacokinetics of TAF Compositions in Dogs

Provided compositions were administered via single subcutaneous (SC) injection to male beagle dogs and were evaluated as described in Example 9. Each formulation tested in this Example comprised 7.8 wt % tenofovir alafenamide sebacate and a vehicle comprising SAIB/PC/PLGA 90-10, 62/28/10). The PLGA 90-10 had varying MWs.

TABLE 15

| | PLGA | | TAF | Detectable | TFV-DP |
|---|---|---|---|---|---|
| | 90-10 | Detectable | plasma | TFV-DP | intracellular |
| Form. | MW | TAF plasma | LLOQ | intracellular | LLOQ |
| No. | (kDa)^{a} | level (days)^{b} | (ng/mL)^{c} | level (days)^{d} | (nM)^{e} |
| F58 | 18 | 33.0 | 0.01 | 54.3 | 10 |
| F59 | 18 | 29.0 | 0.01 | 49.0 | 10 |
| F95 | 13 | 23.0 | 0.01 | 42.0 | 10 |
| F96 | 16 | 15.6 | 0.01 | 30.0 | 10 |
| F97 | 20 | 15.6 | 0.01 | 36.0 | 10 |

^{a}Measured molecular weight.
^{b}Time of last detected TAF plasma concentration; all values are average of 3 dogs.
^{c} Lower limit of quantification for TAF plasma concentration determination by LC-MS/MS assay.
^{d}Time of last detected intracellular TFV-DP concentration in PBMCs; all values are average of 3 dogs.
^{e}Lower limit of quantification for intracellular TFV-DP concentration in PBMCs determined by LC-MS/MS assay
LLOQ of cell counting assay was 2.0 million cells/sample.

### Example 11. Preparation of Small Molecule Formulations for Alzheimer's Disease

Provided compositions were prepared according to the following general procedure: A small molecule agent for treatment of Alzheimer's disease was added to a vehicle composition (prepared as described in Example 1), followed by homogenization. Before being combined with the vehicle, the small molecule agent had a d90 particle size of about 45 microns. The formulations described in Table 16 were prepared according to the above procedure. In Table 16, % active loading (w/w) is based on free base equivalence values.

TABLE 16

| Form. | | % Active | Solution or | |
|---|---|---|---|---|
| No. | Active | Loading (w/w) | Suspension | Vehicle |
| A1 | Free Base | 8.5 | Solution | V90 |
| A2 | Free Base | 8.5 | Suspension | V91 |
| A3 | Free Base | 8.5 | Suspension | V92 |
| A4 | Free Base | 8.5 | Suspension | V93 |
| A5 | Free Base | 8.5 | Suspension | V94 |
| A6 | Free Base | 8.5 | Solution | V95 |
| A7 | Free Base | 8.5 | Suspension | V96 |
| A8 | Free Base | 8.5 | Suspension | V97 |
| A9 | Free Base | 8.5 | Suspension | V98 |
| A10 | Free Base | 8.5 | Suspension | V99 |
| A11 | Free Base | 8.5 | Solution | V100 |
| A12 | Free Base | 8.5 | Solution | V101 |
| A13 | Free Base | 8.5 | Solution | V102 |
| A14 | Free Base | 8.5 | Solution | V103 |
| A15 | Free Base | 8.5 | | V104 |
| A16 | Free Base | 8.5 | | V105 |
| A31 | Tosylate | 8.5 | | V106 |
| A32 | Tosylate | 8.5 | Suspension | V107 |
| A34 | Tosylate | 8.5 | | V108 |
| A35 | Tosylate | 8.5 | Suspension | V109 |
| A36 | Tosylate | 8.5 | Solution | V110 |
| A42 | Tosylate | 8.5 | | V111 |

### Example 12. Irradiation Effect on Small Molecule Formulations for Alzheimer's Disease

Injectable formulations are often irradiated to render them aseptic for use. This Example demonstrates the stability of formulations following irradiation. Representative samples were prepared as generally described in Example 11 above with components as summarized in Table 16. Samples were gamma irradiated at 18-21 kGy.

The injection characteristics of the irradiated formulations were investigated. Several formulations were tested for injection time using a 1 mL Exel syringe under 5 lbf, delivering 0.5 mL (nominally) volume and using 21 to 22 G 1″ (UTW) needles. Two syringes were tested per formulation. The results are summarized in Table 17.

TABLE 17

| Form. | Needle | Injection |
|---|---|---|
| No. | (G) | Time (sec) |
| A1 | 21 | 3.3, 2.8 |
| A2 | 21 | 2.8, 3.0 |
| A4 | 21 | 2.6, 2.9 |
| A7 | 22 | 2.0, 2.0 |
| A9 | 22 | 1.9, 2.0 |
| A11 | 21 | 2.8, 3.2 |
| A15 | 21 | 3.2, 3.4 |
| A32 | 21 | 2.4, 2.5 |
| A34 | 21 | 6.1, 6.4 |

The viscosities of the formulations were measured at a shear rate of 50 s⁻¹ to 500 s⁻¹ at 25° C. (n=2). The results are summarized in Table 18.

TABLE 18

| Form. | Viscosity |
|---|---|
| No. | (cP) |
| A1 | 824 |
| A2 | 576 |
| A4 | 669 |
| A7 | 98 |
| A9 | 62 |
| A11 | 748 |
| A15 | 771 |
| A32 | 578 |
| A34 | 1528 |

### Example 13. In Vitro Release of Small Molecule Formulations for Alzheimer's Disease

The present disclosure provides formulations that allow for prolonged release when administered (e.g., via injection). This Example provides representative embodiments of formulations that achieve prolonged release.

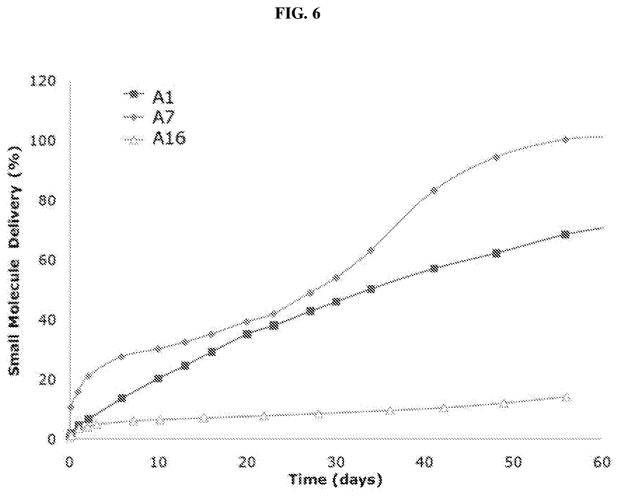

Representative samples were prepared as generally described in Example 11 above with components as summarized in Table 16. The release of active agent from the samples was measured in aqueous buffer (pH˜4.7+0.5% SDS (ammonium acetate) at 37° C.). Briefly, 0.3 mL of sample formulation (room temperature) was placed in a fixed surface area cup, which was moved to 100 mL fresh 37° C. buffer at each time point; gentle agitation was performed during release rate testing. The cumulative release (%) of the small molecule from selected formulations is plotted in FIG. 6.

### Example 14. PK of Small Molecule Formulations for Alzheimer's Disease in Dogs

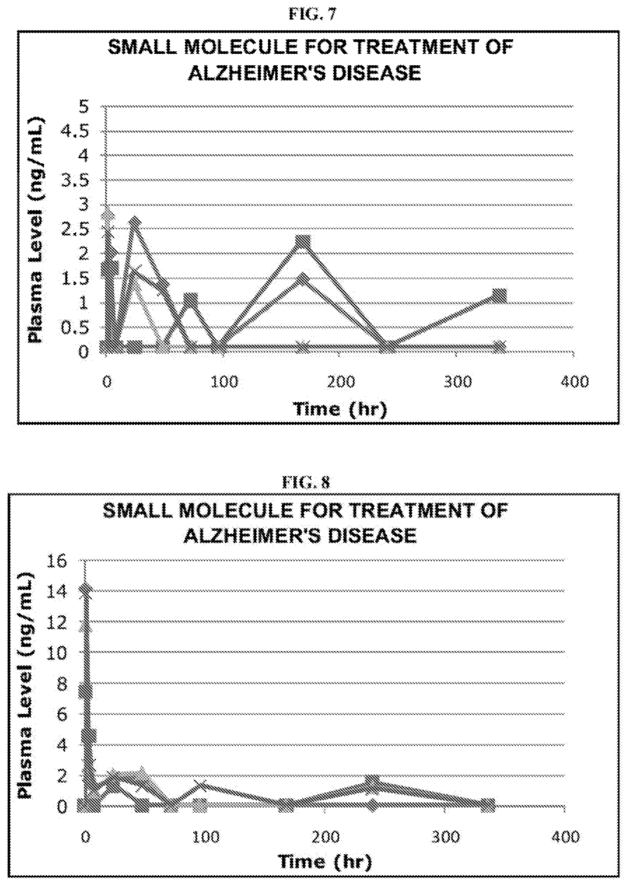

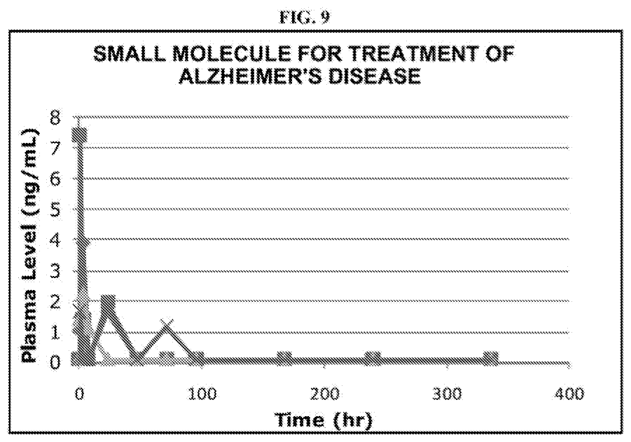

Provided compositions were administered via single subcutaneous (SC) injection of 0.5 mL of provided composition to male beagle dogs. FIG. 7 shows the PK profile of the small molecule for treatment of Alzheimer's disease in dogs from formulation A1, a composition comprising sucrose acetate isobutyrate, N-methyl-pyrrolidone, and a poly(lactic acid)(glycolic acid) (PLGA). FIG. 8 shows the PK profile of the small molecule for treatment of Alzheimer's disease in dogs from formulation A7, a composition comprising sucrose acetate isobutyrate, propylene carbonate, and a 75:25 poly(lactic acid)(glycolic acid) (PLGA). FIG. 9 shows the PK profile of the small molecule for treatment of Alzheimer's disease in dogs from formulation A16, a composition comprising sucrose acetate isobutyrate, propylene carbonate, and a 90:10 poly(lactic acid)(glycolic acid) (PLGA).

### Example 15. Preparation of Risperidone Formulations

Provided compositions were prepared according to the following general procedure: SAIB was heated in a 60° C. oven. Solvent was weighed into a container with a stir bar. PLGA was added to the solvent with stirring until dissolution was achieved. Heated SAIB was added and stirred until a homogeneous composition was obtained.

After the vehicle was prepared, the water content of the vehicle was immediately measured. The jar containing the vehicle was nitrogen flushed at 6000 cc/min overnight at ambient condition. The water content was measured again to ensure ˜0.15% water content was achieved.

In other cases, after the vehicle was prepared, the water content of the vehicle was immediately measured. Calculated amount of water was added to the jar and stirred for 20-30 minutes. The water content was measured again to ensure ˜0.6% water content was achieved.

In still other cases, after the vehicle was prepared, the water content of the vehicle was immediately measured. Calculated amount of water was added to the jar and stirred for 20-30 minutes. The water content was measured again to ensure ˜0.9% water content was achieved.

Risperidone was added to vehicle compositions, followed by homogenization. Before being combined with the vehicle, the risperidone typically had a particle size of approximately 5 microns (irregular shape).

The formulations described in Table 19 were prepared according to the above procedure.

TABLE 19

| | | | Vehicle | |
|---|---|---|---|---|
| | | RSP | % Water | |
| For- | | Loading | Content | Physical |
| mulation | Vehicle Composition | (wt %) | (nominal) | Form |
| R1 | SAIB/PC/90-10 PLGA (19 kDa) (62/28/10) | 10% | 0.15 | Suspension |
| R2 | SAIB/PC/90-10 PLGA (19 kDa) (62/28/10) | 10% | 0.6 | Suspension |
| R3 | SAIB/PC/90-10 PLGA (19 kDa) (62/28/10) | 10% | 0.9 | Suspension |
| R4 | SAIB/NMP/75-25 PLGA (8 kDa) (50/30/20) | 10% | 0.15 | Suspension |
| R5 | SAIB/NMP/75-25 PLGA (8 kDa) (50/30/20) | 10% | 0.6 | Suspension |
| R6 | SAIB/NMP/75-25 PLGA (8 kDa) (50/30/20) | 10% | 0.9 | Suspension |

Each PLGA was initiated with 1-dodecanol.

### Example 16. Irradiation Effect on Risperidone Formulations

Injectable formulations are often irradiated to render them aseptic for use. This Example demonstrates the stability of formulations following irradiation. Formulations were prepared as described in Example 15 above with components as summarized in Table 19. Samples were gamma irradiated at nominally 15 kGy (15.9 kGy to 17.5 kGy) or nominally at 28 kGy to 35 kGy (28.6 kGy to 31.5 kGy).

The water content of the formulations was tested and shown in Table 20.

TABLE 20
T = 0, Non-irradiated

| | Wt % Water | |
|---|---|---|
| | | Reported Average |
| Formulation | Nominal | (n = 2) |
| R1 | 0.15 | 0.21 |
| R2 | 0.6 | 0.50 |
| R3 | 0.9 | 0.72 |
| R4 | 0.15 | 0.32 |
| R5 | 0.5 | 0.62 |
| R6 | 0.9 | 0.90 |

T = 0, 15 kGy irradiated

| | Wt % Water | |
|---|---|---|
| | | Reported Average |
| Formulation | Nominal | (n = 2) |
| R1 | 0.15 | 0.22 |
| R2 | 0.5 | 0.46 |
| R3 | 0.9 | 0.64 |
| R4 | 0.15 | 0.32 |
| R5 | 0.5 | 0.64 |
| R6 | 0.9 | 0.93 |

T = 0, 30 kGy irradiated

| | Wt % Water | |
|---|---|---|
| | | Reported Average |
| Formulation | Nominal | (n = 2) |
| R1 | 0.15 | 0.19 |
| R2 | 0.5 | 0.47 |
| R3 | 0.9 | 0.64 |
| R4 | 0.15 | 0.32 |
| R5 | 0.5 | 0.73 |
| R6 | 0.9 | 1.08 |

The potency of formulations at T=0, after storage for 3 months at 25° C., or after 3 months at 40° C. was tested. Results are shown in Table 21.

TABLE 21

| | | | % RSP (w/w) | | |
|---|---|---|---|---|---|
| For- | Nominal | | | T = 3 | T = 3 |
| mulation | Water | Irradiation | T = 0 | m 25 C. | m 40 C. |
| R1 | 0.15% | 0 kGy | 11.5 | 10.7 | 9.4 |
| | | 15 kGy | 10.5 | 11.2 | 9.9 |
| | | 28-35 kGy | 11.1 | 11.2 | 9.9 |
| R2 | 0.6% | 0 kGy | 10.8 | 10.9 | 10.0 |
| | | 15 kGy | 10.8 | 11.7 | 11.2 |
| | | 28-35 kGy | 10.1 | 10.7 | 10.7 |
| R3 | 0.9% | 0 kGy | 11.6 | 11.4 | 10.0 |
| | | 15 kGy | 11.0 | 12.4 | 10.3 |
| | | 28-35 kGy | 9.3 | 10.8 | 9.1 |
| R4 | 0.15% | 0 kGy | 10.0 | 11.6 | 9.1 |
| | | 15 kGy | 9.8 | 10.8 | 9.4 |
| | | 28-35 kGy | 9.3 | 10.5 | 9.2 |
| R5 | 0.6% | 0 kGy | 10.6 | 10.6 | 9.8 |
| | | 15 kGy | 10.0 | 10.5 | 9.2 |
| | | 28-35 kGy | 9.1 | 11.2 | 9.4 |
| R6 | 0.9% | 0 kGy | 8.8 | 11.2 | 9.6 |
| | | 15 kGy | 9.7 | 11.0 | 10.3 |
| | | 28-35 kGy | 8.7 | 12.3 | 10.1 |

The potency data of these suspension formulations may be not representative due to the inhomogeneity of the formulations and difficulty of sampling from the formulation vials.

The total degradation of formulations at T=0, after storage for 3 months at 25° C., or after 3 months at 40° C. was tested. Results are shown in Table 22.

TABLE 22

| | | | | 3 months at | 3 months at |
|---|---|---|---|---|---|
| | | | T = 0 | 25 C. | 40 C. |
| For- | Nominal | | % Total | % Total | % Total |
| mulation | Water | Irradiation | Deg | Deg | Deg |
| R1 | 0.15% | 0 kGy | 0.02 | 0.04 | 0.18 |
| | | 15 kGy | 0.05 | ND | 0.17 |
| | | 28-35 kGy | 0.05 | 0.03 | 0.15 |
| R2 | 0.6% | 0 kGy | 0.03 | 0.05 | 0.18 |
| | | 15 kGy | 0.06 | ND | 0.14 |
| | | 28-35 kGy | 0.06 | 0.03 | 0.12 |
| R3 | 0.9% | 0 kGy | 0.03 | 0.04 | 0.20 |
| | | 15 kGy | 0.06 | 0.04 | 0.14 |
| | | 28-35 kGy | 0.07 | 0.04 | 0.14 |
| R4 | 0.15% | 0 kGy | 0.05 | 0.17 | 0.58 |
| | | 15 kGy | 0.09 | 0.03 | 0.12 |
| | | 28-35 kGy | 0.11 | 0.02 | 0.11 |
| R5 | 0.6% | 0 kGy | 0.02 | 0.17 | 0.48 |
| | | 15 kGy | 0.09 | 0.03 | 0.14 |
| | | 28-35 kGy | 0.10 | 0.03 | 0.16 |
| R6 | 0.9% | 0 kGy | 0.06 | 0.21 | 0.55 |
| | | 15 kGy | 0.10 | 0.03 | 0.13 |
| | | 28-35 kGy | 0.13 | 0.02 | 0.13 |

For T=0, the irradiated samples exhibited a slightly higher level of total degradation products (TDP) than the control samples (0 kGy) indicating the irradiation effect, however, the water content did not show the significant impact on degradation for both formulations.

For samples stored 3 months at 25° C., the TDP decreased for the irradiated samples but increased for the control samples (slightly for the PC formulations while much more for the NMP formulations).

For samples stored 3 months at 40° C., the TDP increased for both formulations, and the control samples had a the higher TDP than the irradiated samples, most significantly for the NMP formulations.

The polymer molecular weight of formulations at T=0, after storage for 3 months at 25° C., or after 3 months at 40° C. was tested. Results are shown in Table 23.

TABLE 23

| | | | | | 3 months at | | 3 months at | | 3 mo | 3 mo |
|---|---|---|---|---|---|---|---|---|---|---|
| | | | T = 0 | | 25 C. | | 40 C. | | retest | retest |
| | Nominal | | PLGA | | | % of | % of | | (25 C.) | (40 C.) |
| | Water | | Mw | % of | Mw | T = 0, | Mw | T = 0, | Mw | Mw |
| Form. | Content | Irradiation | (Da) | 0 kGy | (Da) | 0 kGY | (Da) | 0 kGY | (Da) | (Da) |
| R1 | 0.15% | 0 kGy | 19186 | 100 | 17041 | 88.82 | 6574 | 34.27 | | |
| | | 15 kGy | 17576 | 91.6 | 15582 | 81.22 | 6573 | 34.26 | | |
| | | 28-35 kGy | 17354 | 90.5 | 14220 | 74.12 | 6485 | 33.80 | | |
| R2 | 0.6% | 0 kGy | 19286 | 100 | 11549 | 59.88 | 831 | 4.31 | | |
| | | 15 kGy | 18074 | 93.7 | 9796 | 50.79 | 834 | 4.32 | | |
| | | 28-35 kGy | 17120 | 88.8 | 9114 | 47.26 | 787 | 4.08 | | |
| R3 | 0.9% | 0 kGy | 19025 | 100 | 8964 | 47.12 | 802 | 4.22 | 8415 | |
| | | 15 kGy | 17714 | 93.1 | 7490 | 39.37 | 787 | 4.14 | 7015 | |
| | | 28-35 kGy | 16926 | 89 | 7635 | 40.13 | 822 | 4.32 | 7174 | |
| R4 | 0.15% | 0 kGy | 9353 | 100 | 9261 | 99.02 | 4482 | 47.92 | 8996 | 4415 |
| | | 15 kGy | 8963 | 95.8 | 7385 | 78.96 | 3593 | 38.42 | 7154 | 3502 |
| | | 28-35 kGy | 8719 | 93.2 | 7957 | 85.08 | 5272 | 56.37 | 7873 | 5090 |
| R5 | 0.6% | 0 kGy | 9405 | 100 | 7343 | 78.08 | 927 | 9.86 | | |
| | | 15 kGy | 9007 | 95.8 | 6813 | 72.44 | 850 | 9.04 | | |
| | | 28-35 kGy | 8709 | 92.6 | 5726 | 60.88 | 841 | 8.94 | | |
| R6 | 0.9% | 0 kGy | 9403 | 100 | 6242 | 66.39 | 804 | 8.55 | 6041 | |
| | | 15 kGy | 8960 | 95.3 | 4896 | 52.07 | 796 | 8.47 | 4489 | |
| | | 28-35 kGy | 8622 | 91.7 | 5314 | 56.52 | 755 | 8.03 | 5010 | |

Overall, MW data support that lower irradiation dose is better and water content less than 0.35% is better.

### Example 17. In Vitro Release of Risperidone Formulations

The present disclosure provides formulations that allow for prolonged release when administered (e.g., via injection). This Example provides representative embodiments of formulations that achieve prolonged release.

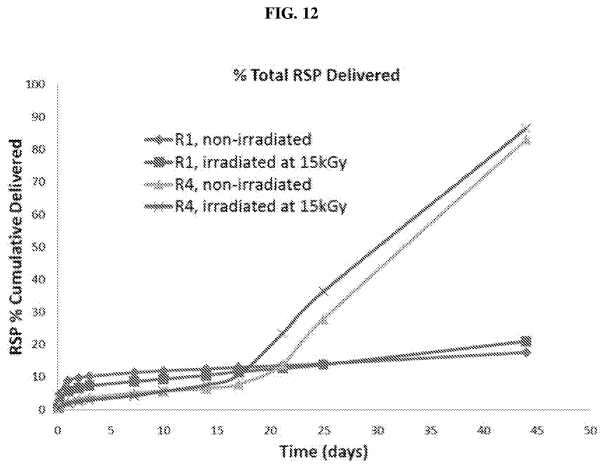

Representative samples were prepared as generally described in Example 15 above with components as summarized in Table 19. The release of active agent from the samples was measured in aqueous buffer (Dulbecco's PBS, pH 7.4) at 37° C. Briefly, 0.5 mL of sample formulation (room temperature) was placed in a fixed surface area cup, which was moved to 100 mL fresh 37° C. buffer at appropriate time intervals; gentle agitation was performed during release rate testing. The cumulative release (%) of the small molecule from selected formulations is plotted in FIG. 12.

### Example 18. Preparation of Naltrexone Formulations

Provided compositions were prepared according to the following general procedure: SAIB was heated in a 60° C. oven. Solvent was weighed into a container with a stir bar. PLGA was added to the solvent with stirring until dissolution was achieved. Heated SAIB was added and stirred until a homogeneous composition was obtained.

After the vehicle was prepared, the water content of the vehicle was immediately measured. The jar containing the vehicle was nitrogen flushed at 6000 cc/min overnight at ambient condition. The water content was measured again to ensure ˜0.15% water content was achieved.

In other cases, after the vehicle was prepared, the water content of the vehicle was immediately measured. Calculated amount of water was added to the jar and stirred for 20-30 minutes. The water content was measured again to ensure ˜0.8% water content was achieved.

Naltrexone was added to vehicle compositions, followed by homogenization. Before being combined with the vehicle, the naltrexone typically had a particle size of approximately 50 microns to 260 microns (rod-like crystals).

The formulations described in Table 24 were prepared according to the above procedure.

TABLE 24

| | | NTX | % Water | |
|---|---|---|---|---|
| For- | | Loading | Content | Physical |
| mulation | Vehicle Composition | (wt %) | (nominal) | Form |
| N1 | SAIB/BA/75-25 | 3% | 0.15 | solution |
| | PLGA (11 kDa) | | | |
| | (70/22/8) | | | |
| N2 | SAIB/BA/75-25 | 3% | 0.8 | solution |
| | PLGA (11 kDa) | | | |
| | (70/22/8) | | | |
| N3 | SAIB/PC/90-10 | 5% | 0.15 | sus- |
| | PLGA (19 kDa) | | | pension |
| | (62/28/10) | | | |
| N4 | SAIB/PC/90-10 | 5% | 0.8 | sus- |
| | PLGA (19 kDa) | | | pension |
| | (62/28/10) | | | |

Each PLGA was initiated with 1-dodecanol.

### Example 19. Irradiation Effect on Naltrexone Formulations

Injectable formulations are often irradiated to render them aseptic for use. This Example demonstrates the stability of formulations following irradiation. Formulations were prepared as described in Example 18 above with components as summarized in Table 24. Samples were gamma irradiated at nominally 15 kGy (16.5 kGy to 18.2 kGy) or nominally at 28 kGy to 35 kGy (29.3 kGy to 32.3 kGy).

The water content of the formulations was tested and shown in Table 25 (“0 kGy” indicates a control formulation that was not irradiated).

TABLE 25

| | wt % |
|---|---|
| | Water |
| | Content |
| Batch/Group | T = 0 |
| N1, 0.15% H2O, 0 kGy | 0.14 |
| N1, 0.15%H2O, 15 kGy | 0.15 |
| N1, 0.15%H2O, 28-35 kGy | 0.15 |
| N2, 0.8% H2O, 0 kGy | 0.74 |
| N2, 0.8% H2O, 15 kGy | 0.73 |
| N2, 0.8% H2O, 28-35 kGy | 0.73 |
| N3, 0.15% H2O, 0 kGy | 0.10 |
| N3, 0.15% H2O, 15 kGy | 0.11 |
| N3, 0.15% H2O, 28-35 kGy | 0.10 |
| N4, 0.8% H2O, 0 kGy | 0.68 |
| N4, 0.8% H2O, 15 kGy | 0.67 |
| N4, 0.8% H2O, 28-35 kGy | 0.67 |

The potency of formulations at T=0, after storage for 11 days at 60° C., 28 days at at 40° C. or at 60° C., or after 42 days at 5° C. or 40° C. was assayed. Results are shown in Table 26.

TABLE 26

| | | | Potency | | | | | |
|---|---|---|---|---|---|---|---|---|
| | Nominal | Irradiation | | 11 | 28 | 28 | 42 | 42 |
| | Water | Dose | | days | days | days | days | days |
| Form. | (wt %) | (kGy) | T = 0 | 60 C. | 60 C. | 40 C. | 40 C. | 5 C. |
| N1 | 0.15 | 0 | 99.0 | 96.3 | 96.0 | 104.2 | 95.0 | Not |
| | | 15 | 96.7 | 95.7 | 94.7 | 100.8 | 94.3 | tested |
| | | 30 | 95.3 | 94.0 | 92.7 | 98.6 | 92.0 | |
| N2 | 0.8 | 0 | 100.3 | 98.3 | 93.0 | 98.0 | 96.3 | Not |
| | | 15 | 98.3 | 97.7 | 91.3 | 97.0 | 94.7 | tested |
| | | 30 | 97.0 | 95.7 | 88.3 | 95.7 | 93.0 | |
| N3 | 0.15 | 0 | 131.8 | 111.8 | 104.2 | 104.4 | 101.8 | 102.8 |
| | | 15 | 132.2 | 109.6 | 99.2 | 103.0 | 92.8 | 101.6 |
| | | 30 | 130.0 | 112.0 | 99.8 | 100.6 | 100.0 | 101.4 |
| N4 | 0.8 | 0 | 108.0 | 102.0 | 94.4 | 97.0 | 101.8 | 102.2 |
| | | 15 | 107.2 | 70.8 | 92.2 | 95.0 | 100.0 | 102.8 |
| | | 30 | 101.8 | 80.6 | 92.2 | 93.7 | 92.8 | 91.6 |

The T=0 and 11 days at 60° C. data of the suspension formulations (N4 and N3) were not representative due to the inhomogeneity of the formulations and difficulty of sampling from the formulation vials. The assay was done using a whole vial assay for the suspension samples of 28 and 42 days.

For the solution formulations (N1 & N2), there appears to be a decrease in potency with increasing irradiation dose; however, there is significant analytical variability in the potency data.

For the suspension formulations (N3 & N4), there is significant analytical variability in the potency data, limiting the conclusions that can be drawn.

The total degradation products of formulations at T=0, after storage for 11 days at 60° C., 28 days at at 40° C. or at 60° C., or after 42 days at 5° C. or 40° C. was assayed. Results are shown in Table 27.

TABLE 27

| | | | Total Degradation Products (%) | | | | | |
|---|---|---|---|---|---|---|---|---|
| | Nominal | Irradiation | | 11 | 28 | 28 | 42 | 42 |
| | Water | Dose | | days | days | days | days | days |
| Form. | (wt %) | (kGy) | T = 0 | 60 C. | 60 C. | 40 C. | 40 C. | 5 C. |
| N1 | 0.15 | 0 | 0.57 | 1.23 | 3.19 | 1.63 | 2.22 | — |
| | | 15 | 1.04 | 1.29 | 2.92 | 1.33 | 1.92 | — |
| | | 30 | 1.57 | 1.92 | 3.40 | 2.12 | 2.35 | — |
| N2 | 0.8 | 0 | 0.53 | 1.21 | 3.63 | 1.74 | 2.03 | — |
| | | 15 | 1.14 | 1.45 | 3.36 | 1.47 | 1.68 | — |
| | | 30 | 1.81 | 1.77 | 3.70 | 1.92 | 2.47 | — |
| N3 | 0.15 | 0 | ND | ND | ND | ND | ND | ND |
| | | 15 | 0.21 | ND | ND | ND | ND | 0.25 |
| | | 30 | 0.25 | ND | ND | 0.23 | ND | 0.35 |
| N4 | 0.8 | 0 | ND | ND | 0.79 | ND | 0.10 | ND |
| | | 15 | 0.22 | ND | 0.63 | ND | ND | 0.24 |
| | | 30 | 0.37 | ND | 0.65 | ND | ND | 0.41 |

At t=0, total degradation products increased with increasing irradiation dose for both solution (N1 & N2) and suspension (N3 & N4) formulations. Water content did not have a significant impact on total degradation products at t=0.

For the solution formulations (N1 & N2), total degradation products increased significantly with storage time; however, there did not appear to be significant effects of irradiation dose or water content on the observed levels of total degradation products.

For the suspension formulations (N3 & N4), there did not appear to be signicant effects of storage time, irradiation dose or water content on the reported levels of total degradation products.

The polymer molecular weight of formulations at T=0, after storage for 11 days at 60° C., 28 days at at 40° C. or at 60° C., or after 42 days at 40° C. was assayed. Results are shown in Table 28.

TABLE 28

| | | Irrad. | PLGA MW | | | | | | | | | | | |
|---|---|---|---|---|---|---|---|---|---|---|---|---|---|---|
| | Water | Dose | T = 0 | | 11 days 60 C. | | 28 days 60 C. | | 28 days 40 C. | | 42 days 40 C. | | 42 d 40 C. (repeat) | |
| Form. | (wt %) | (kGy) | MW | % MW | MW | % MW | MW | % MW | MW | % MW | MW | % MW | MW | % MW |
| N1 | 0.15 | 0 | 9616 | 100.0 | 773 | | 757 | | 803 | | 796 | | | |
| | | 15 | 8449 | 87.9 | 784 | | 760 | | 800 | | 749 | | | |
| | | 30 | 8076 | 84.0 | 785 | | 763 | | 801 | | 735 | | | |
| N2 | 0.8 | 0 | 8911 | 100.0 | 757 | | 756 | | 784 | | 755 | | | |
| | | 15 | 7867 | 88.3 | 757 | | 757 | | 784 | | 740 | | | |
| | | 30 | 7484 | 84.0 | 757 | | 758 | | 783 | | 785 | | | |
| N3 | 0.15 | 0 | 19677 | 100.0 | 16718 | 85.0 | 10323 | 52.5 | 18936 | 96.2 | 18786 | 95.5 | 17926 | 91.1 |
| | | 15 | 18373 | 93.4 | 14809 | 75.3 | 9656 | 49.1 | 16944 | 86.1 | 15003 | 76.2 | 15601 | 79.3 |
| | | 30 | 17421 | 88.5 | 14515 | 73.8 | 9632 | 49.0 | 15923 | 80.9 | 15635 | 79.5 | 14615 | 74.3 |
| N4 | 0.8 | 0 | 19405 | 100.0 | 4884 | 25.2 | 768 | 4.0 | NT | NA | 8374 | 43.2 | | |
| | | 15 | 18162 | 93.6 | 4827 | 24.9 | 772 | 4.0 | 9215 | 47.5 | 6692 | 34.5 | | |
| | | 30 | 17149 | 88.4 | 4273 | 22.0 | 773 | 4.0 | 8340 | 43.0 | 6149 | 31.7 | | |

% MW is the MW of the PLGA as a percentage relative to PLGA MW at t = 0, 0 kGy.

The PLGA MW of the solution formulations N2 and N1 degraded significantly after storage for 11 days at 60° C. and 28 days at 60° C. and 40° C. The PLGA peak was merged with the SAIB peak in the GPC chromatograms.

The PLGA degraded significantly faster in solutions formulations than the suspension formulations (N3 & N4).

### PLGA MW Results: Solution and Suspension Formulations, T=0

A negative effect of irradiation on the PLGA MW was observed: the higher irradiation doses resulted in the lower PLGA MW. The irradiation effect (% MW) on the PLGA MW was similar between formulations N2 and N1. The irradiation effect (% MW) was also similar between formulations N4 and N3, indicating that the initial change in PLGA MW was not impacted by the water content at T=0. The irradiation effect was higher on the solution formulations (N1 & N2) than the suspension formulations (N3 & N4).

PLGA MW Results: Suspension Formulations N4 and N3, T=0 to 42 days

Formulation N4 (0.8% water) had significantly higher PLGA degradation than formulation N3 (0.15% water) after storage for 28 days at 60° C. and 40° C., indicating the negative effect of the water content. At 40° C., the irradiation effect was observed at 28 and 42 days: the higher irradiation dose resulted in the higher PLGA MW degradation.

Overall, the MW data supports that water content less than 0.35% is better and lower irradiation dose is better for PLGA MW stability.

The viscosity of several formulations was measured at 150 s⁻¹ with results shown in Table 29.

TABLE 29

| | Irradiation | |
|---|---|---|
| | Dose | Viscosity |
| Formulation | (kGy) | (cP)^{a} |
| N1 | 0 | 743 |
| N1 | 15 | 785 |
| N1 | 30 | 803 |
| N2 | 0 | 689 |
| N2 | 15 | 683 |
| N2 | 30 | 683 |
| N3 | 0 | 1610 |
| N3 | 15 | 1470 |
| N3 | 30 | 1442 |
| N4 | 0 | 1235 |
| N4 | 15 | 1183 |
| N4 | 30 | 1155 |

^{a}Average of two measurements except for N4 , 15 kGy

### Example 20. In Vitro Release of Naltrexone Formulations

The present disclosure provides formulations that allow for prolonged release when administered (e.g., via injection). This Example provides representative embodiments of formulations that achieve prolonged release.

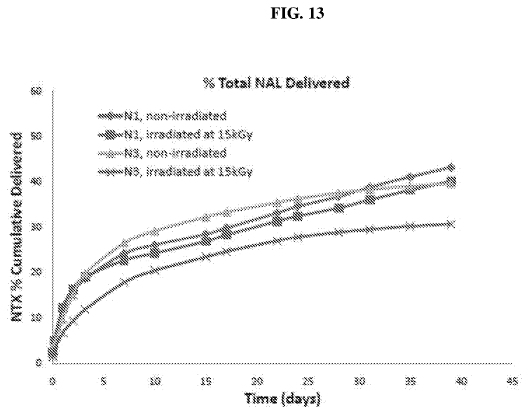

Representative samples were prepared as generally described in Example 18 above with components as summarized in Table 24. The release of active agent from the samples was measured in aqueous buffer (Dulbecco's PBS, pH 7.4) at 37° C. Briefly, 0.5 mL of sample formulation (room temperature) was placed in a fixed surface area cup, which was moved to 50 mL fresh 37° C. buffer at appropriate time intervals; gentle agitation was performed during release rate testing. The cumulative release (%) of the small molecule from selected formulations is plotted in FIG. 13.

### INCORPORATION BY REFERENCE

All publications, patents, and patent applications mentioned herein are hereby incorporated by reference in their entirety.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A composition comprising:
an active agent;
a non-polymeric, non-water soluble high viscosity liquid carrier material (HVLCM) having a viscosity of at least 5000 cP at 37° C. that does not crystallize neat at 25° C. and 1 atmosphere;
a lactic acid-based polymer;
a solvent; and
0.001 wt % to 0.35 wt %, based on total weight of the composition, of water.

2. The composition of claim 1, wherein the HVLCM comprises sucrose acetate isobutyrate.

3. The composition of claim 2, wherein the composition comprises from about 25 wt % to about 70 wt % sucrose acetate isobutryate, based on the total weight of the composition.

4. The composition of claim 2, wherein the composition comprises from about 40 wt % to about 60 wt % sucrose acetate isobutyrate, based on the total weight of the composition.

5. The composition of claim 1, wherein the lactic acid-based polymer comprises poly(lactic acid)(glycolic acid).

6. The composition of claim 5, wherein the poly(lactic acid)(glycolic acid) comprises lactic acid repeat units and glycolic acid repeat units in a molar ratio from about 70:30 to about 100:0, respectively.

7. The composition of claim 5, wherein the poly(lactic acid)(glycolic acid) comprises lactic acid repeat units and glycolic acid repeat units in a molar ratio from about 65:35 to about 95:5, respectively.

8. The composition of claim 1, wherein the lactic acid-based polymer has a weight average molecular weight from about 5 kDa to about 25 kDa when measured using gel permeation chromatography.

9. The composition of claim 5, wherein the poly(lactic acid)(glycolic acid) has a weight average molecular weight from about 15 kDa to about 55 kDa when measured using gel permeation chromatography.

10. The composition of claim 5, wherein the composition comprises from about 5 wt % to about 30 wt % poly(lactic acid)(glycolic acid), based on the total weight of the composition.

11. The composition of claim 1, wherein the composition comprises from about 5 wt % to about 20 wt % lactic acid-based polymer, based on the total weight of the composition.

12. The composition of claim 1, wherein the solvent comprises propylene carbonate.

13. The composition of claim 1, wherein the composition comprises from about 10 wt % to about 40 wt % solvent, based on the total weight of the composition.

14. The composition of claim 1, wherein the composition comprises from about 20 wt % to about 60 wt % propylene carbonate, based on the total weight of the composition.

15. The composition of claim 1, wherein the composition comprises from about 5 wt % to about 30 wt % active agent, based on the total weight of the composition.

16. The composition of claim 1, wherein the composition comprises, based on the total weight of the composition:
(i) from about 5 wt % to about 15 wt % active agent;
(ii) from about 5 wt % to about 10 wt % poly(lactic acid)(glycolic acid);
(iii) from about 20 wt % to about 30 wt % propylene carbonate; and
(iv) from about 50 wt % to about 60 wt % sucrose acetate isobutyrate.

17. The composition of claim 1, wherein the composition has been stored for at least 1 week.

18. The composition of claim 1, wherein the composition has been sterilized, optionally wherein the composition has been sterilized by gamma irradiation.

19. A method of manufacturing the composition of claim 1, comprising:
(a) providing an active agent; and
(b) combining the active agent, with HVLCM, lactic acid-based polymer, and solvent to form the composition.

20. A method of administering a therapeutically effective amount of an active agent, comprising administering to a subject the composition of claim 1.
